(19) United States
(12) Patent Application Publication
(10) Pub. No.: US 2024/0081703 A1
(43) Pub. Date: Mar. 14, 2024

(54) BLOOD COLLECTION DEVICES AND METHODS

(71) Applicant: Health Made Easy, Beverly Hills, MI (US)

(72) Inventor: Richard Penington, Beverly Hills, MI (US)

(21) Appl. No.: 18/367,630

(22) Filed: Sep. 13, 2023

Related U.S. Application Data
(60) Provisional application No. 63/406,050, filed on Sep. 13, 2022.
(60) Provisional application No. 63/406,059, filed on Sep. 13, 2022.
(60) Provisional application No. 63/406,074, filed on Sep. 13, 2022.
(60) Provisional application No. 63/406,061, filed on Sep. 13, 2022.
(60) Provisional application No. 63/406,067, filed on Sep. 13, 2022.

(51) Int. Cl. A61B 5/15

(52) U.S. Cl. CPC A61B 5/150984; A61B 5/150022; A61B 5/150389; A61B 5/150755

(57) ABSTRACT

The invention provides devices for blood collection that integrate a plurality of microneedles for an easy-to-use, pain-free blood collection experience. The invention provides both plasma and whole blood collection and separation in a single device. The invention provides for the collection, storage, and maintenance of blood specimens for stable transport to diagnostic laboratories, in varying ambient temperatures and time durations. Specifically, the invention provides a blood collection device that allows a user to collect an accurately metered quantity of blood.

### CROSS-REFERENCE TO RELATED APPLICATIONS

This application claims priority to, and the benefit of, U.S. Provisional Application No. 63/406,050, filed Sep. 13, 2022, U.S. Provisional Application No. 63/406,059, filed Sep. 13, 2022, U.S. Provisional Application No. 63/406,061, filed Sep. 13, 2022, U.S. Provisional Application No. 63/406,067, filed Sep. 13, 2022, and U.S. Provisional Application No. 63/406,074, filed Sep. 13, 2022, the content of each of which is incorporated by reference herein in its entirety.

### FIELD OF THE INVENTION

The invention relates to direct-to-consumer devices and methods for blood collection.

### BACKGROUND

The ability to collect accurate blood samples is an important component in disease diagnosis and monitoring, and critical to providing quality healthcare. Typical blood collection via venipuncture can pose challenges. For example, the reliance on phlebotomists contributes to overall laboratory medicine and health system costs. Patients must present themselves to a laboratory, and entering a clinic introduces opportunity for infectious disease transmission. Further, approximately 25% of adults have a fear of needles, which can lead to avoidance due to the physical and/or emotional discomfort of the blood draw experience.

Additionally, the burden of frequent in-person monitoring for venous blood draws contributes to disengagement from prophylactic and other care. For certain patient populations, the lack of access to, or difficulty in accessing, providers as well as the burden of in-person testing is associated with health risks.

In an effort to provide accessibility, several advances in remote or at-home blood sample collection have facilitated the collection of blood samples, including serial collection. Remote or at-home blood sample collection offers advantages such as compliance with necessary testing and improved accessibility. Patients can also save time by collecting a sample at home and then sending it to a lab for analysis.

### SUMMARY

Conventional at-home devices are designed with usability in mind, however, limitations still exist. Home collection devices are typically bulky and complicated, and require a finger prick using a lancet. Often operator error results in poor quality or insufficient collection. Further, finger prick sampling can be painful and may not produce a sufficient volume of blood to perform required testing. Repeated finger pricks of the same area can cause sensitivity and must be done correctly to produce high-quality blood samples. Thus, use of these devices has been challenged by technical issues related to difficulty of use, poor quality of sample collection, insufficient quantity collected, and low levels of patient participation.

The invention provides devices for blood collection. Specifically, the invention provides blood collection devices that allows a user to collect a metered quantity of blood that is stored within the device for use by a diagnostic testing laboratory. Devices of the invention allow blood collection at any point of care, including but not limited to home, office, clinic, a kiosk, in the field or in any public or private setting.

Blood collection devices of the invention comprise integrated features, such as for example, a microneedle assembly, a collection well, a capillary microstructure, and one or more fill zones. The device may be housed on a solid support or as a flexible and deformable support designed for placing on a patch of skin on the body. The support optionally includes a plurality of layers to achieve the functionality of the devices, and hydrophobic (e.g., wax) or physical (e.g., cut or embedded polymer) barriers to define features of the devices. In addition, devices of the invention may include an analgesic, an antiseptic, an anticoagulant, and/or a meter to monitor collection volume. Beneficially, gravity is not required for blood collection using devices of the invention. Accordingly, devices of the invention provide a greater degree of flexibility and accuracy during blood collection, for example by allowing a user to better observe and manage blood collection.

Preferred devices of the invention comprise integrated microneedles to pierce skin and direct a metered quantity of blood, either as whole blood or plasma, to fill zones within the device. The microneedles of the devices are positioned within the device for optimal collection and minimization of pain. The microneedles are aligned such that blood draw and collection are standardized across samples and users, thus reducing variability in quantity and quality of the blood draw. A blood collection well, a capillary microstructure, and a fill indicator, also integrated into the devices, acting in conjunction with the microneedles, ensures that blood flowing from the puncture area is not visible to the user. Thus, the invention provides a uniform puncture that achieves a metered volume of blood, and eliminates operator error and sample contamination issues.

In preferred embodiments, devices of the invention allow the collection, separation, and storage of sufficient quantities of different components of blood, such as white blood cells, red blood cells, platelets, and plasma, in a single device. For example, devices of the invention store the collected blood as whole blood, or the devices are used to separate plasma from whole blood and store each as separate samples on the same device. This allows for a sufficient quantity of blood for multiple diagnostic testing. This is especially necessary for remote locations in areas with limited testing facilities. For example, using blood and plasma collected and separated in a single device, plasma can be used to test for a virus, and then, using the results of the plasma testing, whole blood can be tested to determine the prescription for or treatment of the virus.

Devices of the invention also avoid or minimize hemolysis and provide enhanced blood specimen stability for collection, storage, maintenance, and transport. Devices of the invention provide improved blood specimen stability over a range of ambient conditions for extended durations. Blood samples are also stabilized over varying environmental conditions, including conditions with high ambient humidity, and a varying range of pressures. Again, this is important for remote locations away from local laboratory testing facilities. Thus, the invention integrates all aspects of the blood collection experience into a lightweight and flexible support structure convenient for mailing back to a laboratory for testing. Upon reconstitution, blood samples collected by, and stored within, the device are comparable to samples obtained by venous blood draw or other method of drawing blood for analysis.

The invention provides blood collection devices that are housed in a solid support for collection by insertion of a finger or thumb. The invention also provides for blood collection devices that are housed on a flexible substrate that is placed on the surface of the body for blood collection, such as on a shoulder, forearm, thigh, or buttock. For example, the flexible substrate may resemble an adhesive patch. The device is designed to adhere to skin during the blood collection process to ensure complete collection of a sufficient quantity of blood. Thus, the invention provides for flexibility in where blood is collected to avoid repeatedly piercing the same areas, and also allows for easier and less painful collection of blood from the elderly and critically ill, as well as infants and children.

In one aspect, the invention provides a blood collection device comprising a plurality of microneedles disposed on a support; at least one fill zone disposed within the support, the fill zone capable of receiving and holding a quantity of blood; and a capillary microstructure extending through the support and capable of conducting the blood from the microneedles into the fill zone. In some embodiments, the capillary microstructure comprises lateral distribution channels.

Devices of the invention are structured around the novel use of microneedles as the piercing mechanism for the blood draw. The microneedles can be hollow and/or solid. Additionally, in some embodiments, the microneedles may be coated with an analgesic to reduce the pain of the piercing, and/or an anticoagulant to minimize or prevent clotting.

The invention includes devices with the microneedles and piercing mechanism integrated into the structure of the device and retained within the device. In other arrangements, the microneedles and piercing mechanism are housed in a standalone piercing apparatus. In still 10 other configurations, the microneedles are housed in a piercing apparatus integrated into the device, but which can be removed from the device after blood collection is complete. In each embodiment of the devices, the microneedles are placed to standardize the locus of the blood draw, minimize pain from the piercing, ensure uniformity of the blood sample collected, and to ensure that a sufficient quantity of blood is consistently drawn across populations and piercing 15 sites.

In some embodiments, the support is configured to allow a user to place a finger in contact with the device for a time sufficient to allow the fill zone to receive a desired quantity of blood. In general configurations, for microneedles integrated into devices designed to collect blood from a finger or thumb, the microneedles may be arranged as an array housed within the device, for example in a concave cradle on the surface of the device. For example, the support of the device may be configured to allow a user to insert a finger into the device and hold the finger in place until the fill zones receive a desired quantity of blood. In some embodiments, the devices of the invention are configured such that the finger is held in place by a surface that conforms to the finger and guides a placement of the finger for piercing by the microneedles.

In some embodiments, the support is a substantially flat surface. In some embodiments, the support is flexible to allow conformation to a surface to which it is applied. In some embodiments, the support comprises a substrate. For example, the substrate the substrate is a hydrophilic, porous media for receiving blood, in some embodiments. In some embodiments, the support further includes one or more layers, and one or more barriers disposed on the one or more layers. For example, in some embodiments, the barriers define one or more of a sample addition zone, the capillary microstructure, the fill zone, and the fill indicator. As discussed in more detail herein, in some embodiments, the barrier is a hydrophobic material. In some embodiments, the one or more layers define a top surface and a bottom surface.

In other aspects, the invention provides blood collection devices that are housed in a solid support for collection by insertion of a finger or thumb. When the user places the finger in the cradle, the microneedles are actuated to pierce the finger. The user then holds the finger there until blood collection is complete. Further, the user's finger may be held in place by a surface that conforms to the finger and guides placement of the finger for piercing by the microneedles. For example, the cradle may have a deformable substrate to help hold the finger in place during the blood collection process. This allows for a uniform and uninterrupted collection of the specimen to eliminate operator error and sample contamination issues.

For flexible devices designed for blood collection from body surface areas other than 10 fingers, the microneedles may be arranged as a plurality of arrays within the device. The microneedles are actuated to pierce the skin once the device has been placed on the skin. The microneedles may be actuated by applying pressure to the device, for example, by pressing on the device with the palm of a user's hand.

The microneedles may also be in a piercing apparatus that is attached to the surface of the 15 blood collection device, for example shaped as a button that can be removed upon completion of the blood collection process. For example, the compartment may be attached via a snap or latch, or by a tab. In some configurations, the microneedles are actuated to pierce the skin by pressing on the button. The microneedles then retract back into the compartment once blood collection is complete. The compartment can then be removed from the device and thrown away. For example 20 the compartment may be removed by unlatching, unsnapping, or twisting off the compartment.

Devices of the invention also include microneedles as part of standalone piercing apparatuses used to pierce skin and collect blood which is applied to the blood collection well of the device. The apparatuses are designed for piercing a finger, such as the ring or middle finger, a thumb, or a toe. The standalone piercing apparatus may be configured, for example, as a cradle that can lay flat on a surface for a user to easily place a finger into for piercing by the microneedles. The cradle may include a deformable material to help hold the finger in place while blood is collected. The standalone piercing apparatus of devices of the invention may be shaped, for example, as a thimble, and designed to pierce any digit.

The invention also provides for the microneedles as a standalone piercing apparatus that is a lancet for dropping blood into a variety of blood collection devices. The standalone piercing apparatuses of the invention provide the advantage of sufficient, reduced-pain, high-quality blood collection from any digit and the flexibility of placing blood into any device. The standalone piercing apparatuses of the invention may also include a capillary tube for extraction of blood from the apparatus for adding to devices of the invention.

In preferred embodiments, devices of the invention and/or the piercing apparatuses provide for retracting the microneedles back into the piercing apparatus or the device itself after blood collection is complete. In this way, the biohazard waste element is removed so the device can be mailed to the diagnostic testing laboratory.

In addition to the microneedles as the piercing mechanism, devices of the invention also include, in various forms, integrated components such as an analgesic, a blood collection well for capturing a volume of blood, an anticoagulant, a capillary microstructure extending through the support of the device for directing the blood through the device to fill zones, and a fill indicator for indicating when a sufficient quantity of blood has been collected. The presence of an analgesic is especially beneficial in alleviating the amount of pain experienced by the user of the device. The alleviation of pain also results in increased compliance for using the device and collecting the blood samples.

Blood is collected in the collection well by the integrated microneedles or from the microneedle piercing apparatus. From the blood collection well, the blood enters the capillary microstructure to separate the blood components and fill the respective fill zones.

In some embodiments, the devices of the invention comprise a separation mechanism that retains a cellular fraction of the blood at a predetermined location on the support and conducts plasma to the fill zone. In some embodiments, the separation mechanism is selected from the capillary microstructure, a gel, filter paper, and a particle.

In some embodiments, the capillary microstructure comprises a plurality of layers. The capillary microstructure of the devices may be contained within layers of the support and defined by, for example, hydrophobic barriers. In certain embodiments, the hydrophobic barrier may be a hydrophobic wax barrier. The hydrophobic wax barriers also define the blood collection well or sample addition zone, the fill zones, and the fill indicator. The layers of the support allow for the separation of the components the blood collection into discrete fill zones. For example, layers of the device may include a top and bottom, a laminate layer on a top portion of the device for sealing, layers of polyester mesh, clear polyethylene terephthalate (PET) mylar membranes, polyester membranes, clear PETG membranes, asymmetrical polysulfone membranes, double-sided adhesive to affix the membranes, and a cellulose or chromatography paper layer or cut out. The blood collection well may include a substrate that is a hydrophilic, porous media for receiving blood.

Blood collection using devices of the invention can be driven by vacuum pressure. In some embodiments, the devices of the invention include a vacuum source sufficient to draw blood from a needle puncture site to the capillary microstructure. In one example, the capillary microstructure functions as a closed system creating a passive vacuum between the point of 5 extraction and the capillary microstructure. The vacuum can be created by forming a seal at the point of entry to the capillary microstructure. In a preferred configuration, the capillary microstructure is air-free. The capillary microstructure may also contain vents and valves that provide and/or maintain a vacuum between the collection needles and the capillary microstructure. Regulation of the vents and valves is used to control vacuum and flow into the 10 capillary microstructure. In one example, the capillary microstructure may be a microfluidic assembly made by, for example multilayer soft lithography processes that incorporate valves and vents into the microfluidic chambers. Finally, the device may comprise an active pump or other mechanism to create a vacuum and thus promote flow of blood from the needle assembly into the capillary microstructure.

Devices of the invention include fill zones for the collection of the blood specimen. The fill zones receive a quantity of whole blood, separated plasma, or any other component of the blood, conducted from the blood collection well by the capillary microstructure. Again, the capillary microstructure, in conjunction with the layers of the support can separate components of the whole blood, such as plasma, and direct plasma and whole blood into separate fill zones on the same device. The capillary microstructure may direct the blood or plasma to the respective fill zones via lateral distribution channels defined by barriers within the device (which can be physical barriers and/or hydrophobic barriers). In some embodiments, the devices include a fill indicator that visibly shows when the fill zone has received a pre-determined quantity of blood.

Devices of the method include a novel metered fill indicator. The fill indicator is a visible indicator that a sufficient quantity, such as a pre-determined quantity, of blood for testing and analysis, has been collected. The user can easily determine when the blood collection process is complete. The fill indicator gives a visual indication to the user that the correct quantity of blood has been received into the fill zones and prevents overfilling or underfilling of the zones. Thus, the fill indicators of the device ensure a metered quantity of blood is collected in the fill zones which rejection of the sample at the diagnostic testing laboratory for insufficient quantity.

The invention provides devices with a flexible configuration of fill zones. For example, certain devices of the invention allow for the separation of the blood into different components such as white blood cells, red blood cells, platelets, and plasma, and direct the components to specific fill zones within the device. Thus, the device delivers metered whole blood volumes, metered plasma volumes, and/or metered amounts for any other component of the blood, for flexible testing options. The device may be configured to have a ratio of whole blood fill zones to plasma fill zones depending on the blood tests required. This flexibility allows for an improved array of diagnostic tests possible for the specimen collected, an important feature for areas lacking local and easily accessible laboratory services.

The invention provides blood collection devices that are housed in a solid support for collection by insertion of a finger or thumb, in some embodiments. When the user places the finger in the cradle, the microneedles are actuated to pierce the finger. The user then holds the finger there until blood collection is complete. Further, the user's finger may be held in place by a surface that conforms to the finger and guides placement of the finger for piercing by the microneedles. For example, the cradle may have a deformable substrate to help hold the finger in place during the blood collection process. This allows for a uniform and uninterrupted collection of the specimen to eliminate operator error and sample contamination issues.

Aspects of the invention provide blood collection devices as described above specifically with a cradle for positioning a finger or thumb of a user on the device such that the finger or thumb is guided to engage with the microneedles of the device. The device for collection of a blood sample may include a plurality of microneedles disposed on a support, at least one fill zone disposed within the support, the fill zone capable of receiving and holding a quantity of blood, a capillary microstructure extending through at least a portion of the support and capable of conducting the blood from the microneedles into the fill zone; and a cradle positioning a finger or a thumb of the person at a predetermined position on the device.

In certain preferred embodiments, the invention provides blood collection devices comprising a plurality of microneedles disposed on a support, and a compartment attached to the support, wherein the compartment positions the digit at a predetermined position on the device. In certain aspects of the invention, the compartment for positioning the finger is a cradle. Accordingly, the invention provides devices comprising a plurality of microneedles disposed on a support, at least one fill zone disposed within the support, wherein the fill zone is capable of receiving and holding a quantity of blood, a capillary microstructure, and a cradle positioning a finger or a thumb of the person at a predetermined position on the device. More specifically, the predetermined position on the device is on top of the microneedles. The compartment and/or cradle is especially advantageous to place the digits of the users, irrespective of their height, weight, age, digit length, or digit girth, to place the digits at an optimal position for consistent drawing of blood.

The aforementioned compartment and/or cradle may be attached to the support holding the microneedles. The cradle is designed to guide the engagement of the digit of the user to the plurality of microneedles. The digit may be a finger or thumb of the user. The cradle is designed for positioning a digit of a user on the device such that the digit is guided to engage with the microneedles of the device. The digit may be a finger, a thumb, or a toe of the user. The cradle is beneficial in consistent placement of the digit of the user at the same position on the device, which leads to a uniform blood draw for the users of the device, independent of the dimensions of the digit of the user. The compartment is designed to guide engagement of the digit with the microneedles, and to hold the digit steady during the blood collection process. Moreover, the consistent placement is also important to reduce the pain experienced by the user of the device. In particular, the placement may be such that the digit is guided on the device so that the nerve centers in the digits are positioned away from the microneedles, which results in reduced pain for the user. This is also beneficial in increasing the compliance of the users.

It is ideal to have a consistent blood draw across different users to reduce sample variability and have more consistent quantitative analysis. Another advantage of consistent blood draw is that a user would be assured that a requisite amount of sample is drawn from the finger which avoids the user having to submit the sample again due to lack of volume of blood required for any test.

The compartment and/or cradle may be prepared from a material such as plastic or elastic film, a deformable polymer, foam, or a combination of these materials. These compartments and/or cradles may have a tubular or conical shape, designed to guide the digit of the user to the top of microneedles. The cradle may also be comprised of a deformable or elastic material that deforms upon application of force by a digit of the user. In certain embodiments of the invention, the cradle may further include an attachment to hold the digit of the user on the predetermined position on the device for a duration in which the requisite amount of blood is collected. The attachment is particularly advantageous as it may reduce the instances where the required amount of blood is not collected from the user because the user may flinch upon contact with the microneedle and withdraw the digit prior to collection of the required amount of blood.

As mentioned above, the cradle is designed to guide a digit of the user to a predetermined position on the device, which is on top of the microneedles. Thus, the invention provides that the cradle has openings such that the digit of the user may contact the microneedles. These openings may be holes aligned with the microneedles such that the microneedles can be deployed through the holes to pierce the skin of the finger or thumb held within the cradle. These holes would be aligned such that when the digit of the user is optimally positioned, the microneedles go through the holes and contact the skin for blood collection. In another embodiment, the opening may be in a shape of a circular disk on top of the microneedles. This opening is important so that there is maximal contact between the digit and the microneedles resulting in optimal blood collection.

The microneedles may be mounted on a spring mechanism to actuate deployment of the microneedles. The actuation may be application of a force on the cradle by the finger or thumb. This is advantageous in avoiding the accidental deployment of microneedles so that they are deployed only when the digit of the user is optimally positioned for blood collection. The actuation mechanism may be a spring-loaded mechanism which is actuated by the application of force. The actuation may also happen by pressing a button. Relatedly, the microneedles retract into the initial position once the force is removed and/or the blood collection is complete. The retraction of the microneedles after the sample collection ensures there is no biohazard in shipping the device after the blood collection is complete. The microneedles may also be in a piercing apparatus that is attached to the surface of the blood collection device, for example shaped as a button that can be removed upon completion of the blood collection process. For example, the compartment may be attached via a snap or latch, or by a tab. In some configurations, the microneedles are actuated to pierce the skin by pressing on the button. The microneedles then retract back into the compartment once blood collection is complete. The compartment can then be removed from the device and thrown away. For example the compartment may be removed by unlatching, unsnapping, or twisting off the compartment.

The cradle of the device may have a thickness of about from about 0.0625 inch to about 0.5 inch. More specifically, the cradle may have a thickness of about 0.25 inches. The width of the cradle may be from about 0.01 inch to about 1 inch, or preferably, it is about 0.5 inch.

Devices of the invention include fill zones for the collection of the blood specimen. The fill zones receive a quantity of whole blood, separated plasma, or any other component of the blood, conducted from the blood collection well by the capillary microstructure. Again, the capillary microstructure, in conjunction with the layers of the support can separate components of the whole blood, such as plasma, and direct plasma and whole blood into separate fill zones on the same device. The capillary microstructure may direct the blood or plasma to the respective fill zones via lateral distribution channels defined by physical barriers.

The cradle may be a material such as plastic or elastic film, a deformable polymer, foam, or a combination of materials. The cradle may have holes aligned with the microneedles such that the microneedles can be deployed though the holes to pierce the skin of the finger or thumb held within the cradle. The microneedles may be mounted on a spring mechanism to actuate deployment of the microneedles. The actuation may be application of a force on the cradle by the finger or thumb. Relatedly, the microneedles may retract into the initial position once the force is removed. The cradle attached to the blood collection device enables the users of the device to place digits consistently on top of the microneedles at an optimal position to provide consistent collection blood. This aspect is especially beneficial because the invention provides consistent blood collection for users with varying digit sizes, especially for users of different age, and having digits with varying dimensions.

These devices may also have a compartment attached to the support to position a digit, such as a finger, thumb, or toe, within the compartment for piercing by the microneedles. The compartment is designed to guide engagement of the digit with the microneedles, and to hold the digit steady during the blood collection process. This is achieved by the compartment being a deformable material, such as a polymer, which conforms to the shape of the digit when the digit is pressed into the compartment. The cradle may have holes aligned with the microneedles such that the microneedles can be deployed though the holes to pierce the skin of the finger or thumb held within the cradle. The microneedles may be mounted on a spring mechanism to actuate deployment of the microneedles. The actuation may be application of a force on the cradle by the finger or thumb. Relatedly, the microneedles retract into the initial position once the force is removed. The compartment is especially advantageous to place the digits of the users, irrespective of their height, weight, age, digit length, or digit girth, at an optimal position for consistent drawing of blood.

Aspects of the invention provide blood collection devices as described above, specifically a flexible embodiment of the support with an adhesive layer for securing to a patch of skin. The blood collection device for collection of blood from a subject includes a plurality of microneedles disposed on a flexible support; at least one fill zone capable of receiving and holding a quantity of blood; a capillary microstructure extending through at least a portion of the support and capable of conducting the blood from the microneedles into the fill zone; and an adhesive pad comprising an adhesive layer for securing the device to skin.

The flexible support may be a stretchable and/or deformable material to conform to the shape of the area to which it is applied. The microneedles are arranged as a number of arrays within the support and activated by a spring mechanism to pierce the skin, wherein the activation of the spring mechanism inserts the plurality of microneedles in the skin of the subject. In some embodiments, the spring mechanism is activated upon application of the device on the skin of the subject. In other embodiments, the spring mechanism is activated upon pressing a button. In other embodiments, the array of microneedles is activated by pressing a button. Further, in some embodiments, upon completion of collection of blood, the microneedles are retracted in the flexible support.

In some embodiments, the fill zones may be housed in a removable subportion of the support for easy release from the adhesive backing. In some embodiments, the removable subportion is bar-coded to identify a patient.

In some embodiments, the device further comprises a removable layer covering the adhesive layer, wherein the removable layer is removed prior to extraction.

In related embodiments, the invention provides a blood collection device with a flexible support as described above with a microneedle piercing apparatus or microneedle member that is releasably attached to the flexible support. The device includes a blood collection channel in fluid communication with the microchannels for directing the blood to the capillary microstructure of the device. The attachment mechanism allows the piercing apparatus to be removed from the device without damaging the blood collection channel or introducing contaminants into the blood collection process. Releasing the piercing apparatus creates a vent to assist in drying the blood specimens collected. The microneedles may be deployed from the piercing apparatus or microneedle member via an actuation mechanism that retracts the microneedles once a predetermined quantity of blood has been collected. The flexible patch provides flexibility to collect blood samples from other parts of the body, including shoulder, legs (including thighs), forearms, buttocks, or back. Thus, in some embodiments, the device is designed for application on the shoulder, biceps, back, hip, buttocks, or leg of the subject.

In preferred embodiments, the invention provides a blood collection device that can be applied as a patch on the body of the user of the device. These devices include a plurality of microneedles disposed on a flexible support. The flexible support has a first side for contacting skin comprising a microneedle assembly comprising a plurality of microneedles and a second side opposite to the first side. The microneedle assembly is designed to contact and pierce the skin to initiate the blood collection. The microneedle assembly may be releasably attached to the flexible support. The devices of the invention further comprise a blood connection channel, wherein the blood connection channel is in fluid communication with the base of at least one microneedle in the microneedle assembly and an attachment mechanism for manually releasing the plurality of microneedles without damaging the blood connection channel.

The blood collection devices are particularly advantageous because they can be applied to several spots on the users' body to collect the optimal amount of blood. For example, the device of the invention may be applied on the shoulder, biceps, back, hip, buttocks, or leg (including thighs) of the user. It is also beneficial to have a releasable microneedle assembly in the patch because the microneedle assembly may be removed from the device prior to mailing the device for analysis. Beneficially, the devices of the invention do not rely on gravity to collect blood from the user. This is important because it can be applied at various locations on body of user in a configuration where the blood collection mechanism does not rely on gravity to collect the blood. For example, it can be used on thigh of user, where the user of the device would be able to observe the collection of the blood from the device. The aspect of user being able to monitor the patch provides several advantages. In particular, the user may be able to analyze if there are any reactions to the device on the skin surrounding the area where the device is applied. The user would also be able to monitor the amount of blood being collected in the device. The removal of microneedle assembly from the device removes the biohazard and allows for safe transfer of the device comprising the collected blood sample for further analysis.

The flexible support of the blood collection device housing the plurality of microneedles is designed to conform to the shape of the body where the blood collection is applied as a patch. The flexible support conforms to the shape of the body such that the plurality of microneedles housed in the flexible patch may contact the skin. The flexible support may comprise a polymer with high elasticity and deformability. The support may vary in thickness to meet the needs of the device, such as about 5000 micrometers or less, in some embodiments from about 1000 to about 2000 micrometers, from about 1 to about 500 micrometers, and in some embodiments, from about 10 to about 200 micrometers. The flexible support may include at least one of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, Gore-Tex, plastic, plastic components, polymer, biopolymer, woven material, non-woven material, and natural material. The flexible support may also comprise a polyurethane-based film. In some embodiments, the flexible support has a shape comprising at least one of a circle, oval, ellipse, square, rectangle, triangle, diamond, butterfly, and hourglass. The invention further provides that the flexible support is larger than the area of microneedle assembly. Thus, there is an area surrounding the microneedle assembly that contains backing with no microneedles. This design makes it easier to apply the device. It also reduces the risk of unintentional contact of the microneedle assembly with the skin either during application or removal. The unintentional contact of the microneedle assembly with the skin can result in bleeding or infection. This is especially important if another person applies or removes the device as this will reduce the risk of transmitting infections between them. The microneedle assembly may be placed in the center of the backing or off center.

Devices of the invention may further comprise an adhesive layer that can help facilitate the attachment of the patch to a user's skin during use. The adhesive layer typically employs an adhesive coated onto a backing material. The backing may be made of a material that is substantially impermeable to blood, such as polymers, metal foils, etc. Suitable polymers may include, for instance, polyethylene terephthalate, polyvinylchloride, polyethylene, polypropylene, polycarbonate, polyester, and so forth. The adhesive may be a pressure-sensitive adhesive. Suitable adhesives may include, for instance, solvent-based acrylic adhesives, solvent-based rubber adhesives, silicone adhesives, etc.

Devices of the invention include the use of a microneedle assembly comprising a plurality of microneedles as the piercing mechanism for the blood draw. The microneedles can be hollow and/or solid. Additionally, the microneedles may be coated with an analgesic to reduce the pain of the piercing, and/or an anticoagulant to minimize or prevent clotting. The microneedles may also be in a piercing apparatus that is attached to the surface of the blood collection device, for example shaped as a button that can be removed upon completion of the blood collection process. For example, the compartment may be attached via a snap or latch, or by a tab. In some configurations, the microneedles are actuated to pierce the skin by pressing on the button. The microneedles then retract back into the compartment once blood collection is complete. The compartment can then be removed from the device and thrown away. For example, the compartment may be removed by unlatching, unsnapping, or twisting off the compartment. The microneedles are arranged as a number of arrays within the support.

The microneedle assembly may be activated by a spring mechanism to pierce the skin. The spring may be activated by application of pressure after application of the patch on the skin of the user. In other embodiments, the spring is actuated by pressing a button, which results in activation of the microneedle assembly. The spring may include a battery spring or coil spring or another spring or component, as long as it can store energy. In other embodiments, the spring may be an air spring, an elastomer, a foam, another fluid spring, a gas spring, another highly compressible material(s), a leaf spring, a sponge, or another member that stores energy (e.g., mechanical or potential energy). The invention further provides that the microneedle assembly may retract in the flexible backing after the blood collection.

Blood collection channels of the invention further comprise a capillary structure to transfer the blood from the plurality of microneedles to the fill zones, wherein each of the fill zones receives and holds a predetermined volume of the blood. Blood is collected in the blood collection well by the integrated microneedles or from the microneedle piercing apparatus. From the blood collection well, the blood enters the capillary microstructure to separate the blood components and fill the respective fill zones. The capillary microstructure of the devices is contained within layers of the support and defined by hydrophobic wax barriers. The hydrophobic wax barriers also define the blood collection well or sample addition zone, the fill zones, and the fill indicator. The layers of the support allow for the separation of the components the blood collection into discrete fill zones. For example, layers of the device may include a top and bottom, a laminate layer on a top portion of the device for sealing, layers of polyester mesh, clear polyethylene terephthalate (PET) mylar membranes, polyester membranes, clear PETG membranes, asymmetrical polysulfone membranes, double-sided adhesive to affix the membranes, and a cellulose or chromatography paper layer or cut out. The blood collection well may include a substrate that is a hydrophilic, porous media for receiving blood.

Fill zones maybe housed in a removable subportion of the device such that the fill zones may be removed from the remaining device after the collection of the blood sample. The removal of fill zones comprising the collected blood is beneficial because of the ease of shipping the collected blood sample for further analysis. Preferably, the removable subportion of the device comprising the fill zone includes an identification tag for identifying the patient. The identification tag may be a QR code, bar code, or a label including the patient's information. The fill zones may be housed in a removable subportion of the support for easy release from the adhesive backing.

In some embodiments, the device further comprises a fill indicator that visibly shows when the fill zone has received a pre-determined quantity of blood.

In some embodiments, the capillary microstructure further provides a separation mechanism that retains a cellular fraction of the blood at a predetermined location on the solid support and conducts plasma to the fill zone.

As disclosed herein, the plurality of microneedles of the blood collection device contain a coating comprising an analgesic and an anticoagulant, in some embodiments. In some embodiments, the flexible support comprises one or more layers, and one or more hydrophobic wax barriers disposed on the one or more layers.

Aspects of the invention provide a blood collection device comprising a flexible material comprising a first side for contacting skin and an opposed second side; a microneedle member releasably attached to the material, the member comprising at least one microneedle positioned to pierce skin when the skin is in contact with the first side; a blood collection channel within the material, wherein a proximal portion of the blood collection channel is in fluid communication with a base of the at least one microneedle; and an attachment mechanism for manually releasing the microneedle member from the material without damaging the blood collection channel.

In some embodiments, the blood collection channel comprises a capillary structure that carries blood away from the at least one microneedle. In some embodiments, a distal portion of the blood collection channel comprises a blood fill zone of a predetermined volume. In some embodiments, the microneedle member displays an exposed base visible on the opposed second side, with a fill indicator visible on the exposed base. In some embodiments, the flexible material comprises a stretchable and/or deformable material to conform to a shape of an area to which the device is applied.

In some embodiments, the attachment mechanism comprises a base structure fixedly attached to the flexible member and wherein the microneedle member is releasably attached to the base structure by a releasable snap-fit or threaded attachment. For example, the blood fill zone is removable from the flexible material, in some embodiments. Further, the blood fill zone is in a covering further comprising a barcode to identify a patient, in some embodiments.

In some embodiments, the first side further comprises an adhesive layer to secure the device to skin. For example, the adhesive layer is covered with a removable adhesive backing and the removable adhesive backing is removed prior to applying the device to skin, in some embodiments.

In some embodiments, the flexible material comprises a stretchable and/or deformable material to conform to a shape of an area to which the device is applied. In some embodiments, the microneedle member is a hollow microneedle. In some embodiments, the microneedle member is attached to an actuation mechanism, wherein the actuation mechanism is actuated for the microneedle member to pierce skin. For example, in some embodiments, the actuation mechanism is a spring-loaded mechanism.

Aspects of the invention provide blood collection devices with a metered fill indicator. The fill indicator is a visible indicator that a sufficient quantity, such as a pre-determined quantity, of blood for testing and analysis, has been collected. The user can easily determine when the blood collection process is complete. The fill indicator gives a visual indication to the user that the correct quantity of blood has been received into the fill zones and prevents overfilling or underfilling of the zones. Thus, the fill indicators of the device ensure a metered quantity of blood is collected in the fill zones which rejection of the sample at the diagnostic testing laboratory for insufficient quantity.

Preferred devices of the invention include a novel fill indicator. A frequent challenge for remote sample collection is the collection of a sufficient quantity of sample for testing. Tests cannot be performed in the laboratory if samples fall short of the minimum quantity of specimen required for testing. Results returned as Quantity Not Sufficient (QNS) mean that less than a minimum required volume or quantity of specimen was received for analyzing the panel of tests ordered. Thus, resampling is required. Additionally, some tests require a specimen for an initial screening, followed by further testing. Confirmatory screening and further testing require the use of another and/or different portions of the original specimen.

The fill indicator ensures that a predetermined quantity of sample, sufficient for the range of tests required, is collected and stored in the device. For example, the fill indicator may provide a visible or other indicator that a sufficient quantity of blood or plasma has been collected. Thus, the fill indicator ensures that a sufficient quantity of blood components required for testing is collected into the device.

The quantity of sample collected may be a pre-determined or metered quantity. The fill indicator may be incorporated into any home or remote sample collection device. For example, the fill indicator is particularly useful for remote sample collection such as with the blood collection devices of the invention. In certain embodiments, the fill indicator is capable of indicating when a metered quantity of sample has been received by the fill zones such that the user knows when the sample collection process is complete.

The blood collection device includes a plurality of microneedles disposed on a support; at least one fill zone disposed within the support, the fill zone capable of receiving and holding a quantity of blood; a capillary microstructure extending through the support and capable of conducting the blood from the microneedles into the fill zone; and a fill indicator disposed on the support.

In some embodiments, the fill indicator indicates a metered quantity of blood has been received by the fill zone from the microneedles. In some embodiments, the fill indicator displays a visible indicator on the support upon collection of a predetermined quantity of blood in the fill zone. For example, in some embodiments, the fill indicator comprises a transparent channel having a first end and a second end, wherein, as blood is conducted from the microneedles into the fill zone, a color indicator fills the channel at the first end and travels toward the second end such that when the fill zone has received a predetermined quantity of blood, the transparent channel undergoes a complete color change to visibly indicate the fill zone has received the predetermined quantity of blood. In some embodiments, the fill indicator comprises an area on the support that appears as a visible checkmark when the fill zone has received a predetermined quantity of blood. In some embodiments, the fill indicator comprises an area on the support in which a word appears to indicate when the fill zone has received a predetermined quantity of blood. In some embodiments, the fill indicator comprises a node disposed on the support such that when a predetermined quantity of blood has been received from the microneedles into the fill zone, the node undergoes a color change. For example, the node is substantially shaped as a button, in some embodiments.

In some embodiments, the fill indicator prevents blood from entering the fill zone once a metered quantity of blood has been received by the fill zone.

Devices of the invention may be configured for a user to insert a finger or other digit into the device and hold the finger or digit in place until the fill zone receives the predetermined quantity of blood. The device may include a structure that conforms to the finger and guides placement of the finger for piercing by the microneedles. The fill indicator thus ensures that a user knows how long to keep the device engaged with the skin before stopping the sample collection process. The user can then easily determine when the blood collection process is complete. The fill indicator gives a visual indication to the user that the correct quantity of blood has been received into the fill zones so that the user can disengage with the device. This prevents overfilling or underfilling of the zones. The predetermined quantity of blood received in the fill zones prevents rejection of the sample at the diagnostic testing laboratory for insufficient quantity.

In certain embodiments using a standalone finger piercing apparatus, the fill indicator may give the user a visual indicator when blood dropped into the collection well has filled the fill zones of the device. The fill indicator indicates when the correct quantity of blood has been received to fill one or multiple fill zones.

The fill indicator may be a transparent channel having a first end as a starting point and a second end as an ending point. When blood is collected in the blood collection well and conducted from the microneedles into the fill zones, a color indicator may fill the channel. The color indicator may fill the indicator at the first end and travel toward the second end as the blood is collected. In this way, the user may watch the color indicator travel through the channel to know when the predetermined quantity of blood has been collected. The color indicator in the channel may undergo a complete color change upon collection of the predetermined quantity of blood. This complete color change further visibly indicates to the user that the blood collection process is complete.

The fill indicator may be one or more areas on the device. The fill indicator may be an area on the device that appears as a visible checkmark when the fill zone(s) has received a predetermined quantity of blood. The fill indicator may be an area on the device in which a word, such as “Filled” appears when the fill zone has received a predetermined quantity of blood.

As noted above, the fill indicator indicates when a metered quantity of blood has been received by the fill zone from the microneedles. The fill indicator may display a visible indicator on the device when the predetermined quantity of blood has been collected.

In other embodiments, the fill indicator may be a node on the support. When a predetermined quantity of blood has been received from the microneedles into the fill zone, the node may undergo a color change. The fill indicator may be a node substantially shaped as a button. The button may be actuated to pop in or pop out when a predetermined quantity of blood has been received.

The device may include a fill indicator for each individual fill zone or for all fill zones collectively. Each fill zone may have its own fill indicator indicating when that fill zone has received a sufficient quantity of blood. Alternatively, the device may have one fill indicator to indicate when the collective zones have each received a predetermined quantity of blood. In some embodiments, the fill indicator indicates when a reservoir of the devices has received a predetermined quantity of blood. In other embodiments, the device may comprise any number of fill indicators to ensure that when a predetermined quantity of blood has been collected, this fact is visibly indicated to the user by the fill indicator.

Importantly, the fill indicator prevents blood from entering the fill zone once a metered quantity of blood has been received by the fill zone.

As disclosed herein, in some embodiments, the microneedles are hollow. In some embodiments the support is adapted to allow a user to insert a finger into the device and hold the finger in contact with the device for a time sufficient to allow the fill zone to receive a desired quantity of blood as indicated by the fill indicator. In some embodiments, the capillary microstructure directs blood to separation media and conducts a cellular fraction of the blood to a predetermined location on the support and conducts plasma to the fill zone. In some embodiments, the support is a substantially flat surface. In other embodiments, the support is flexible to allow conformation to a surface to which it is applied. Further, in some embodiments, the support further includes one or more layers; and one or more barriers disposed on the one or more layers. For example, the barrier is a hydrophobic material, in some embodiments. In some embodiments, the barriers define one or more of a blood collection well, a capillary microstructure, a plurality of fill zones, and a fill indicator. In some embodiments, the one or more layers define a top surface and a bottom surface. In some embodiments, the microneedles contain a coating comprising an analgesic and an anticoagulant.

Aspects of the invention provide methods for collecting blood using the devices of the invention. The methods may include providing a device for collecting blood and collecting blood using the device, the device comprising a plurality of microneedles disposed on a support; at least one fill zone disposed within the support, the fill zone capable of receiving and holding a quantity of blood; a capillary microstructure extending through the support and capable of conducting the blood from the microneedles into the fill zone; and a fill indicator disposed on the support.

In some embodiments of the method, the fill indicator indicates a metered quantity of blood has been received by the fill zone from the microneedles. In some embodiments of the method, the fill indicator displays a visible indicator on the support upon collection of a predetermined quantity of blood in the fill zone.

In some embodiments of the method, the fill indicator comprises a transparent channel having a first end and a second end, wherein, as blood is conducted from the microneedles into the fill zone, a color indicator fills the channel at the first end and travels toward the second end such that when the fill zone has received a predetermined quantity of blood, the transparent channel undergoes a complete color change to visibly indicate the fill zone has received the predetermined quantity of blood. In some embodiments of the method, the fill indicator comprises an area on the support that appears as a visible checkmark when the fill zone has received a predetermined quantity of blood. In some embodiments of the method, the fill indicator comprises an area on the support in which a word appears to indicate when the fill zone has received a predetermined quantity of blood. In some embodiments of the method, the fill indicator comprises a node disposed on the support such that when a predetermined quantity of blood has been received from the microneedles into the fill zone, the node undergoes a color change. In some embodiments of the method, the node is substantially shaped as a button.

In some embodiments of the method, the fill indicator prevents blood from entering the fill zone once a metered quantity of blood has been received by the fill zone.

As disclosed herein, in some embodiments of the method, the microneedles are hollow. In some embodiments of the method, the support is configured to allow a user to insert a finger into the device and hold the finger in place until the fill zone receives the quantity of blood as indicated by the fill indicator. In some embodiments of the method, the capillary microstructure directs blood to separation media and conducts a cellular fraction of the blood to a predetermined location on the support and conducts plasma to the fill zone. In some embodiments of the method, the support is a substantially flat surface. In some embodiments of the method, the support is flexible to allow conformation to a surface to which it is applied. In some embodiments of the method, the support further comprises one or more layers; and one or more barriers disposed on the one or more layers. In some embodiments of the method, the barrier is a hydrophobic material. In some embodiments of the method, the barriers define one or more of a blood collection well, a capillary microstructure, a plurality of fill zones, and a fill indicator. In some embodiments of the method, the one or more layers define a top surface and a bottom surface. In some embodiments of the method, the microneedles are coated with an analgesic and/or an anticoagulant.

Aspects of the invention provide blood collection devices configured to have a ratio of whole blood fill zones to plasma fill zones depending on the blood test required. As described herein, the blood collection device may include a plurality of microneedles disposed on a support; a blood collection well disposed within the support and capable of collecting blood from the microneedles; and a capillary microstructure extending through the support and capable of receiving blood from the blood collection well, separating plasma from blood, and conducting the blood and the plasma into separate fill zones; such that wherein the fill zones are disposed within the support and are capable of receiving and holding a quantity of blood and/or plasma.

For example, the device may include a capillary microstructure housed within a plurality of layers of the support. The capillary microstructure may be arranged to separate plasma from blood and conduct blood and plasma to fill zones on the device in a predetermined ratio. For example, the predetermined ratio of blood fill zones to plasma fill zones arranged on the device may be 0:6, 1:5, 2:4, 3:3, 4:2, 5:1, or 6:0.

Thus, devices of the invention provide metered whole blood volumes, plasma volumes, and/or other component of the blood, for flexible testing options. This flexibility allows for an improved array of diagnostic tests possible for the specimen collected, an important feature for areas lacking local and easily accessible laboratory services. The flexibility of the fill zone configurations also allows for a sufficient quantity of blood for diagnostic testing of more than one test. This is especially necessary for remote locations in areas with limited testing facilities. For example, using blood and plasma collected and separated in a single device, plasma can be used to test for a virus. Then, using the results of the plasma testing, whole blood can be tested to determine the prescription for or treatment of the virus.

Devices of the invention are structured around the novel use of microneedles as the piercing mechanism for the blood draw. The microneedles may also direct a metered quantity of blood, either as whole blood or plasma, to fill zones within the device. The microneedles of the devices are positioned away from pain centers. This allows for a painless or reduced-pain puncture as compared to traditional lancets. In certain embodiments, the microneedles may be placed to standardize the locus of the blood draw. This standardization minimizes pain from the piercing, and ensures uniformity of the blood sample collected. Standardization of the locus also ensures that a sufficient quantity of blood is consistently drawn across populations and piercing sites. Thus, standardizing blood collection across samples and users eliminates variability in quantity and quality of the blood draw.

A blood collection well, a capillary microstructure, and a fill indicator, also integrated into the devices, acting in conjunction with the microneedles, ensure that blood flowing from the puncture area is not visible to the user. Thus, the invention provides for a pleasant, uniform puncture that achieves a metered volume of blood, and eliminates operator error and sample contamination issues.

The microneedles can be hollow and/or solid, or a combination of both. Additionally, the microneedles may be coated with an analgesic to reduce the pain of the piercing, and/or an anticoagulant to minimize clotting.

A blood collection well, a capillary microstructure, and a fill indicator, also integrated into the devices, acting in conjunction with the microneedles, ensures that blood flowing from the puncture area is not visible to the user. Thus, the invention provides for a pleasant, uniform puncture that achieves a metered volume of blood, and eliminates operator error and sample contamination issues.

The devices also avoid or minimize hemolysis and provide enhanced blood specimen stability for collection, storage, maintenance, and transport. Devices of the invention provide improved blood specimen stability over a range of ambient conditions for extended durations. Blood samples are also stabilized over varying environmental conditions, including conditions with high ambient humidity, and a varying range of pressures. Again, this is important for remote locations away from local laboratory testing facilities. Thus, the invention integrates all aspects of the blood collection experience into a lightweight and flexible support structure convenient for mailing back to a laboratory for testing. Significantly, upon reconstitution, blood samples collected by, and stored within, the device are comparable to samples obtained by venous blood draw or other method of drawing blood for analysis.

Aspects of the invention provide for blood collection devices in any of the embodiments described herein. For example, the blood collection device may include a plurality of microneedles disposed on a solid support, a blood collection well disposed within the support 20 and capable of collecting blood from the microneedles, and a capillary microstructure extending through the support. The capillary microstructure is capable of receiving blood from the blood collection well, separating plasma from blood, and conducting the blood and the plasma into separate fill zones. The fill zones are capable of receiving and holding a quantity of blood and/or plasma.

As described herein, in some embodiments, the blood collection well comprises a substrate. For example, in certain embodiments, the substrate is a hydrophilic, porous media. In some embodiments, the blood collection well collects about 150 μL of blood. In some embodiments, the fill zones hold about 10 μL of blood or plasma. In some embodiments, the support is adapted to allow a user to insert a finger in contact with the device for a time sufficient to allow the fill zone to receive a desired quantity of blood or plasma. In some embodiments, the support is a substantially flat surface. In some embodiments, the support is flexible to allow conformation to a surface to which it is applied. In some embodiments, the support further includes one or more layers, and one or more barriers disposed on the one or more layers. For example, the barrier may be a hydrophobic material, in some embodiments. In some embodiments, the barriers define one or more of a blood collection well, a capillary microstructure, a plurality of fill zones, and a fill indicator. In some embodiments, the one or more layers define a top surface and a bottom surface.

In some embodiments, the microneedles are hollow. The microneedles may contain a coating comprising an analgesic and/or an anticoagulant. In certain embodiments, the microneedles are solid. In such case, the device further comprises a vacuum source sufficient to draw blood from a needles puncture site to the capillary microstructure.

As described in detail herein, in some embodiments, the device further includes a fill indicator that visibly shows when the fill zone has received a pre-determined quantity of the blood or plasma.

The blood collection well may include a substrate for receiving the blood, that is, for 5 example, a hydrophilic, porous media. In embodiments, the blood collection well collects about 150 μL of blood and blood is directed to the fill zones such that each fill zone holds about 10 μL of blood or plasma.

Blood may be collected in the collection well by the integrated microneedles or from the microneedle piercing apparatus. From the blood collection well, the blood enters the capillary 10 microstructure to separate the blood components and fill the respective fill zones.

The capillary microstructure of the devices may be contained within a plurality of layers of the support and defined by barriers, for example hydrophobic wax barriers. The hydrophobic wax barriers also define the blood collection well or sample addition zone, the fill zones, and the fill indicator. The layers of the support allow for the separation of the components the blood collection into discrete fill zones. For example, a plurality of layers of the device may include a top and bottom, a laminate layer on a top portion of the device for sealing, layers of polyester mesh, clear polyethylene terephthalate (PET) mylar membranes, polyester membranes, clear PETG membranes, asymmetrical polysulfone membranes, double-sided adhesive to affix the membranes, and a cellulose or chromatography paper layer or cut out.

The blood collection well may include a substrate that is a hydrophilic, porous media for receiving blood. The blood collection well may be coated with the substrate. The substrate may be, for example, a crosslinked hydrogel bound to the blood collection well. The substrate may be functionalized to attract blood into the blood collection well and to the capillary microstructures.

In embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices. The separation mechanism may also be a gel, filter paper or particle.

As disclosed herein, in some embodiments, the invention provides blood collection devices that are housed in a solid support for collection by insertion of a finger or thumb. The invention also provides for blood collection devices that are housed on a flexible substrate that is placed on the surface of the body, other than a finger, for blood collection, such as on a shoulder, forearm, thigh, or buttock. For example, the flexible substrate may resemble an adhesive patch. The device is designed to adhere to skin during the blood collection process to ensure complete collection of a sufficient quantity of blood. Thus, the invention provides for flexibility in where blood is collected to avoid repeatedly piercing the same areas, and also allows for easier and less painful collection of blood from infants and children.

Devices of the invention may be configured for a user to insert a finger or other digit into the device and hold the finger or digit in place until the fill zone receives the predetermined quantity of blood. The device may include a structure that conforms to the finger and guides placement of the finger for piercing by the microneedles.

In embodiments, the devices of the invention include a novel metered fill indicator. The fill indicator ensures that a predetermined quantity of sample, sufficient for the range of tests required, is collected and stored in the device. For example, the fill indicator may provide a visible or other indicator that a sufficient quantity of blood or plasma has been received in the fill zones. testing and analysis has been collected. Thus, the fill indicator ensures that a sufficient quantity of blood components required for testing is collected into the device.

Thus, the blood collection devices of the instant invention provide easy-to-use, reliable, painless, and controlled high-quality blood and plasma separation and collection for better precision and with a quantity sufficient for separation of plasma and whole blood for multiple testing opportunities.

%

### BRIEF DESCRIPTION OF THE DRAWINGS

FIG. 1 is an illustration of a top view of one embodiment of a blood collection device of the invention.

FIG. 2 is an illustration of a bottom view of one embodiment of a blood collection device of the invention.

FIG. 3 illustrates one embodiment of an array of microneedles as integrated into embodiments of the blood collection device of the invention.

FIG. 4 illustrates a top view of one embodiment of a finger prick apparatus of the blood collection device utilizing an array of microneedles positioned within the finger prick apparatus.

FIG. 5 illustrates a perspective view of a finger prick apparatus 400 according to one embodiment of the invention

FIG. 6 illustrates a bottom view of one embodiment of a finger prick apparatus as a cradle housed within the blood collection device.

FIG. 7 illustrates a side view of one embodiment of a finger prick apparatus shaped as a cradle as part of the blood collection device, which utilizes an array of spring-loaded microneedles positioned within the cradle of the finger prick apparatus.

FIG. 8 illustrates a top view of one embodiment of a flexible blood collection device designed to adhere to a user's skin.

FIG. 9 is an illustration of a bottom view of one embodiment of a flexible blood collection device designed to adhere to a user's skin, showing the fill zones for separation of blood and plasma, and integrated microneedles.

FIG. 10 illustrates a side view of one embodiment of a flexible blood collection device of the invention designed to adhere to a user's body.

FIG. 11 illustrates an embodiment of the fill indicator integrated into the device.

FIG. 12 illustrates an embodiment of the device in which the fill indicator is part of a blood reservoir.

FIG. 13 illustrates a side view of one embodiment of the blood collection device of the invention showing the flow of blood from the blood collection well through the fill indicator and reservoir of the device.

FIG. 14 illustrates one embodiment of blood flow from a capillary tube to the blood collection well.

FIG. 15 illustrates one embodiment of the flow of blood from a user's finger to the blood well and into the channel to direct blood to the fill indicator.

FIG. 16 illustrates another embodiment of fill zone configuration in the blood collection device of the invention wherein the blood collection well and plasma fill zones are in a tripod configuration.

FIG. 17 illustrates another embodiment of fill zone configuration in the blood collection device of the invention wherein the blood well and fill zones, both whole blood and plasma zones, are in an angel wing configuration.

FIG. 18 illustrates the flexible embodiments of fill zone configurations of the blood collection device wherein a desired amount of whole blood and/or plasma can be configured by the arrangement of the fill zones.

FIG. 19 illustrates the backside of an embodiment of a flexible device with an adhesive strip and a blood collection well at the center.

### DETAILED DESCRIPTION

The invention provides blood collection devices integrating novel features to achieve a uniform puncture that delivers a metered volume of blood, eliminates operator error and sample contamination issues, and reduces pain associated with the blood draw experience. The invention allows the collection, storage, and maintenance of blood specimens.

Devices of the invention allow simple at-home blood sample collection. For example, a user may use devices of the invention to self-collect a measured quantity of blood at home or from a remote location outside of a clinical setting. The devices also allow for a person other than the user, such as a healthcare provider, parent, or guardian, to collect a blood sample at any time or place on behalf of a person unable to self-collect a sample. The flexibility of devices of the invention allows the collection of an accurately-metered quantity of blood that does not involve a venipuncture. Devices of the invention are useful for providing access to blood testing in areas lacking local laboratory services.

Beneficially, gravity is not required for blood collection using devices of the invention. Accordingly, devices of the invention provide a greater degree of flexibility and accuracy during blood collection, for example by allowing a user to better observe and manage blood collection.

Blood Collection Device

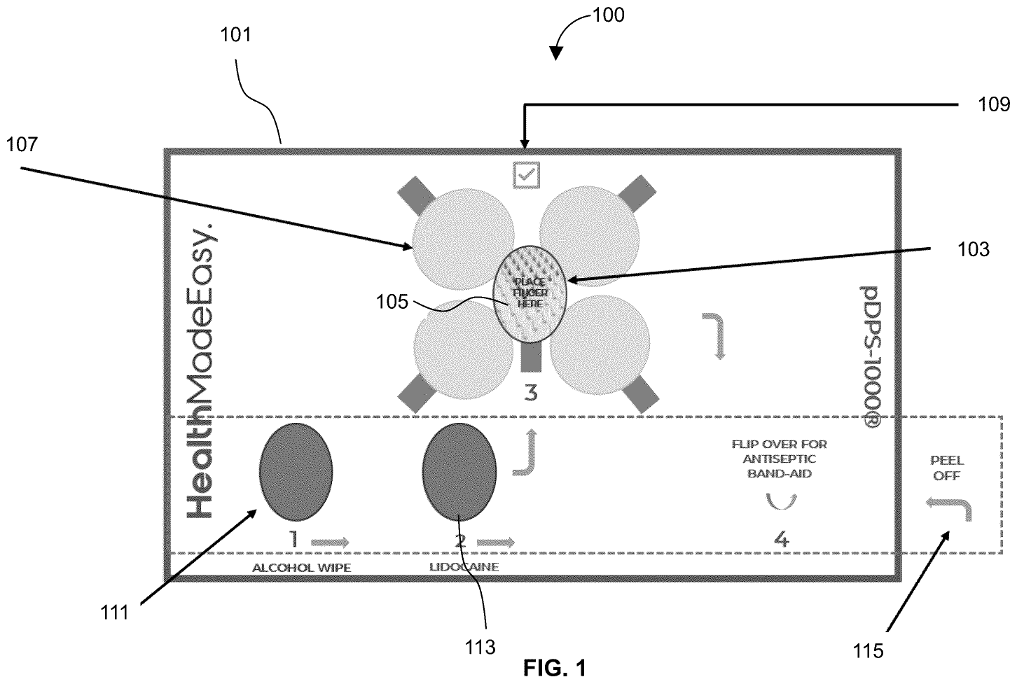

FIG. 1 is an illustration of a top view of one embodiment of a blood collection device 100 of the invention. The blood collection device 100 may integrate all aspects of the blood collection experience into a support 101. The support may be, for example, plastic, cellulose, cardstock, cover stock, pasteboard, paperboard, fiber board, or a cardboard such as a folding boxboard, chipboard, Kraft board, laminated board, or solid bleached or unbleached board. The support may also include a flexible mesh fabric or woven material for flexibility in the application of the device. The support may be one or more of materials that provide the balance of rigidity, flexibility, weight, and durability to house the components of the device.

Aspects of the invention include a blood collection device comprising a plurality of microneedles disposed on a support, at least one fill zone disposed within the support and capable of receiving and holding a quantity of blood, and a capillary microstructure extending through the support and capable of conducting the blood from the microneedles into the fill zone.

As described in more detail below, the support includes a single layer of cardstock impregnated with wax to form the features of the blood collection device, such as a blood collection well 103 housing an array of microneedles 105. The microneedles 105 may be housed, for example, in a concave cradle made of deformable material that conforms to shape of the finger or thumb of the user. The support may include one or more lateral distribution channels (not shown), one or more fill zones 107, and a fill indicator 109. In one example, wax printing may be used to form hydrophobic barriers to control sample flow in the lateral channels to the fill zones. Whole blood may be transported from the blood collection well along lateral channels. In non-limiting examples, blood may be transported via capillary action to the fill zones. The support may include a separation mechanism for separating plasma from whole blood and directing the different component of blood to various fill zones. The separation may be achieved by virtue of the capillary structure within the device.

In some embodiments, the capillary microstructure directs blood to separation media or to a separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper, a foam structure, pillars, or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

Devices of the invention include fill zones for the collection of the blood specimen. The fill zones receive a quantity of whole blood, separated plasma, or any other component of the blood, conducted from the blood collection well by the capillary microstructure. Again, the capillary microstructure, in conjunction with a plurality of layers of the support can separate components of the whole blood, such as plasma, and direct plasma and whole blood into separate fill zones on the same device. The capillary microstructure may direct the blood or plasma to the respective fill zones via lateral distribution channels defined by barriers, for example hydrophobic wax barriers.

The fill zones allow for the collection of blood and/or separated components of blood. The fill zones may comprise pre-punched areas for processing the blood at the testing laboratory. For example, the fill zones may be pre-cut from the main body of the device, such as from a cellulose layer. Thus, removal of the blood sample for processing is facilitated without the need for manual punches, scissors, or cutting tools. The blood contained in the fill zones may also be processed without punching out a fill zone, for example by washing the sample out of the card. This washed-out sample may be further analyzed through in situ testing or PCR. Thus, devices of the invention provide for high-quality sample preparation for a variety of blood tests.

In certain embodiments, the invention provides devices with a flexible configuration of fill zones. For example, the devices of the invention allow for the separation of the blood into different components such as white blood cells, red blood cells, platelets, and plasma, and direct the components to specific fill zones within the device. Thus, the device delivers metered whole blood volumes, metered plasma volumes, and/or metered amounts for any other component of the blood, for flexible testing options. The device may be configured to have a ratio of whole blood fill zones to plasma fill zones depending on the blood tests required. This flexibility allows for an improved array of diagnostic tests possible for the specimen collected, an important feature for areas lacking local and easily accessible laboratory services.

In some embodiments, the device may include an antiseptic in the form of an antiseptic station 111. The antiseptic station may be a cut-out of the support in the form of a depression that may be oval and concave in a shape designed to fit the curvature of the finger or thumb. The antiseptic used in the invention could be any antiseptic for sanitizing the skin prior to puncture or to disinfect the skin after the blood collection is complete. The support may also include an analgesic in the form of an analgesic station 113. The analgesic may also be coated on the microneedles used to pierce the skin and start the blood collection. The analgesic for use in this invention could be any known topically active analgesic, including benzocaine, butamben, dibucaine, lidocaine, oxybuprocaine, pramoxine, proxymetacaine (proparacaine), and tetracaine (amethocaine). The presence of analgesic is especially beneficial in alleviating the amount of pain experienced by the user of the device. The alleviation of pain also results in increased compliance for using the device and collecting the blood samples. The device may also include a cover 115, for example a cellophane wrap, over the stations, as a whole or individually.

The support may also include one or more layers, for example, a laminate layer on a top portion of the device for sealing, layers of polyester mesh, clear PET mylar membranes, polyester membranes, clear PETG membranes, asymmetrical polysulfone membranes, double-sided adhesive to affix the membranes, and a cellulose or chromatography paper layer or cut out. One or more layers may be filter paper or chromatography paper. For example, the paper may be 100% pure cotton linter filter paper such as A-226 paper from Ahlstrom-Munksjo or PerkinElmer, or a general qualitative filter with creped surface such as 226 from Whatman. Alternatively, the filter papers may be Ahlstrom Grade 226, Munktell TFN, and Whatman 903 filter papers.

In some embodiments, the device includes a substrate. For example, the device may include a blood collection well that includes a substrate that is a hydrophilic, porous media for receiving blood. For example, the porous media may be a material such as a porous composite, cellulose, acetate or any other fiber with the required porosity. For example, the substrate may coat aspects of the devices such that the substrate is functionalized to attract blood into the blood collection well, reservoir, or capillary microstructure of the device. The substrate may be, for example, a crosslinked hydrogel bound to the blood collection well.

In some embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper, foam structure or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

The support may include one or more barriers, such as hydrophobic wax barriers, disposed on the one or more layers. The hydrophobic wax barriers may define one or more of a sample addition zone, the capillary microstructure, the fill zone, and the fill indicator. The layers may define a top and a bottom surface of the support.

In certain embodiments, the support may be a patterned dried blood spot support that includes one or more hydrophobic wax barriers disposed on the one or more layers. For example, the wax barriers may be formed by wax printing using a double-sided wax-transfer method to pattern the support. For example, the top and bottom designs may be printed onto laminate sheets, using a wax printer, for example a Xerox ColorQube 8580 wax printer. The various layers, for example a cellulose layer or chromatography paper layer, may be aligned with the top and bottom designs. Alignment can be achieved by a clamp or alignment jig. The wax from the laminate sheets may be transferred to the chromatography paper using a heat press, for example a Promo Heat CS-15, to form the hydrophobic wax barriers. The hydrophobic wax barriers are patterned to define the different parts of the device.

The capillary microstructure may be formed by wax printing, which forms a plurality of hydrophobic barriers to control sample flow in the lateral channels to the fill zones. The capillary microstructure may include lateral distribution channels. The lateral distribution channels may extend throughout the layers of the device to carry whole blood to the various areas of the device for example, for separation and distribution to the fill zones. The lateral channels may be extended past the fill zones to allow for complete saturation of the fill zone for more accurate sampling. The lateral distribution channels may be in fluid communication with the fill zone indicator.

Devices and methods of the invention may separate whole blood samples into two fractions: (i) a first whole blood fraction, which is directed to pre-patterned extraction zone(s) and (ii) a second whole blood fraction, which is directed to a zone that separates plasma from whole blood. This is achieved by using a combination of layers of membranes that separates the different components of blood (i.e., White Blood Cells (WBCs), Red Blood Cells (RBCs), platelets, plasma), and equally distributes the separated plasma into discrete fractions.

The capillary microstructure of the device may provide a separation mechanism that retains a cellular fraction of the blood at a predetermined location on the solid support and conducts plasma to the fill zone.

In some embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

For example, WBCs may be separated by a first membrane such as a polyester membrane. RBCs, cellular debris, and platelets may be separated from the blood sample by a second membrane, for example a polysulfonic membrane. The remaining plasma portion of the sample may be wicked or directed by the capillary microstructure to a fill zone. The fill zone may be a patterned cellulose layer. As detailed below, the invention provides for flexible device configuration such that the fractions of whole blood to plasma collected in the device may vary depending on the needs of the required testing. Thus, the invention delivers both: (i) self-regulated whole blood volumes, and (ii) self-regulated cell-free plasma volumes as a result of the pre-patterned extraction zones that controls sample volume stored in the device, improving analyte quantitation by an improved sampling method.

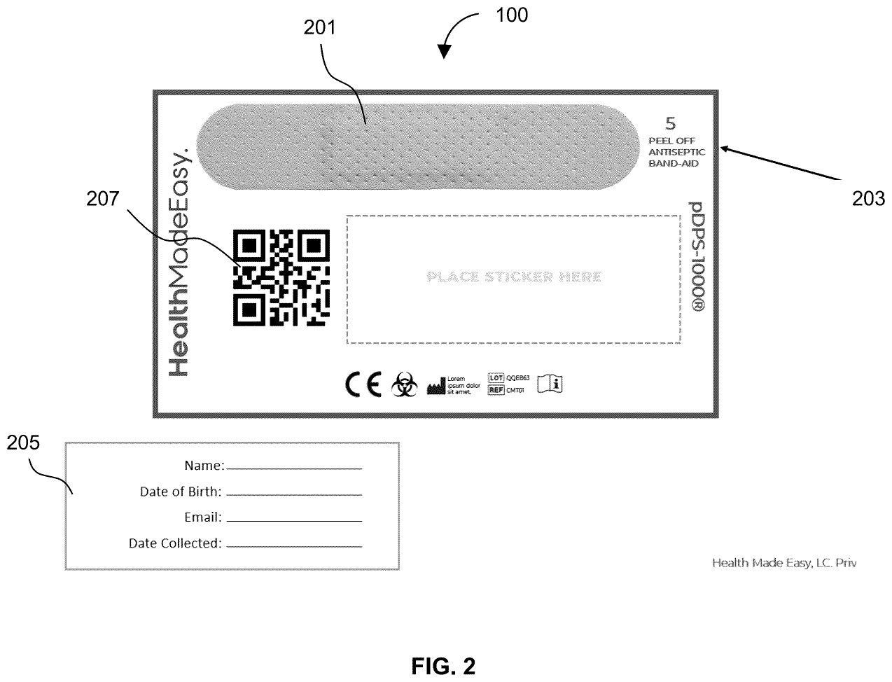

FIG. 2 is an illustration of a bottom view, or back side, of one embodiment of a blood collection device 100 of the invention. The device may include a bandage 201 for applying to the puncture point after the blood collection is complete. The device may include instructions 203 for using the blood collection device printed directly on the device. The instructions may be in the form of numbered steps at stations on the card associated with the blood collection procedure. The device may also include an identification label 205. The device may also include a QR code 207 for entering data regarding the user and the blood collection directly into a database for use by the testing laboratory.

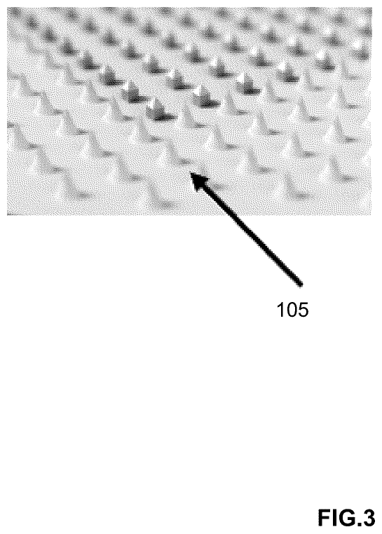

FIG. 3 illustrates one embodiment of an array of microneedles as integrated into embodiments of the blood collection device of the invention. The blood collection device includes a plurality of microneedles 105 to pierce the skin to begin the flow of blood for blood collection into the device. The microneedles may be solid or hollow. Tiny holes made by the needles induce a local change in pressure in the epidermis or dermis that forces blood into a collection device. The needles may be made from a variety of materials, including metals, silicone, polymers, ceramics and glass.

In some embodiments, the microneedles are solid and may include material, for example, filter paper made of cellulose or cotton fiber, surrounding the microneedles to direct the capillary flow of blood from the piercing point of the microneedles to the blood collection well and capillary microstructure of the device. In other embodiments, the microneedles are hollow and direct the flow of blood, for example by capillary action, from the piercing point to the blood collection well and/or the capillary microstructure. The microneedles may contain a coating comprising an analgesic and/or an anticoagulant.

In general configurations, for microneedles integrated into devices designed to collect blood from a finger or thumb, the microneedles may be arranged as an array housed within the device, for example in a concave cradle on the surface of the device. For example, the support of the device may be configured to allow a user to insert a finger into the device and hold the finger in place until the fill zones receive a desired quantity of blood. When the user places the finger in the cradle, the microneedles are actuated to pierce the finger. The user then holds the finger there until blood collection is complete. Further, the user's finger may be held in place by a surface that conforms to the finger and guides placement of the finger for piercing by the microneedles. For example, the cradle may have a deformable substrate to help hold the finger in place during the blood collection process. This allows for a uniform and uninterrupted collection of the specimen to eliminates operator error and sample contamination issues.

For microneedles integrated into flexible devices designed for blood collection from other body surface areas, the microneedles may be arranged as a plurality of arrays within the device. The microneedles are actuated to pierce the skin once the device has been placed on the skin. The microneedles may be actuated by applying pressure to the device, for example, by pressing on the device with the palm of a user's hand.

The microneedles may also be in a piercing apparatus that is attached to the surface of the blood collection device, for example shaped as a button that can be removed upon completion of the blood collection process. For example, the compartment may be attached via a snap or latch, or by a tab. In some configurations, the microneedles are actuated to pierce the skin by pressing on the button. The microneedles then retract back into the compartment once blood collection is complete. The compartment can then be removed from the device and thrown away. For example the compartment may be removed by unlatching, unsnapping, or twisting off the compartment.

Devices of the invention also include microneedles as part of standalone piercing apparatuses used to pierce skin and collect blood which is applied to the blood collection well of the device. The apparatuses are designed for piercing a finger, such as the ring or middle finger, a thumb, or a toe. The standalone piercing apparatus may be configured, for example, as a cradle that can lay flat on a surface for a user to easily place a finger into for piercing by the microneedles. The cradle may include a deformable material to help hold the finger in place while blood is collected. The standalone piercing apparatus of devices of the invention may be shaped, for example, as a thimble, and designed to pierce any digit.

The invention also provides for the microneedles as a standalone piercing apparatus that is a lancet for dropping blood into a variety of blood collection devices. The standalone piercing apparatuses of the invention provide the advantage of sufficient, reduced-pain, high-quality blood collection from any digit and the flexibility of placing blood into any device. The standalone piercing apparatuses of the invention may also include a capillary tube for extraction of blood from the apparatus for adding to devices of the invention.

In certain embodiments, the devices of the invention and/or the piercing apparatuses provide for retracting the microneedles back into the piercing apparatus or the device itself after blood collection is complete. In this way, the biohazard waste element is removed so the device can be mailed to the diagnostic testing laboratory.

In addition to the microneedles as the piercing mechanism, devices of the invention also include, in various forms, integrated components such as an analgesic, a blood collection well for capturing a volume of blood, an anticoagulant, a capillary microstructure extending through the support of the device for directing the blood through the device to fill zones, and a fill indicator for indicating when a sufficient quantity of blood has been collected. The presence of an analgesic is especially beneficial in alleviating the amount of pain experienced by the user of the device. The alleviation of pain also results in increased compliance for using the device and collecting the blood samples.

In a specific aspect of the invention, the invention provides blood collection devices as described above specifically with a cradle for positioning a finger or thumb of a user on the device such that the finger or thumb is guided to engage with the microneedles of the device. The cradle may be a material such as plastic or elastic film, a deformable polymer, foam, or a combination of materials. The cradle may have holes aligned with the microneedles such that the microneedles can be deployed though the holes to pierce the skin of the finger or thumb held within the cradle. The microneedles may be mounted on a spring mechanism to actuate deployment of the microneedles. The actuation may be application of a force on the cradle by the finger or thumb. Relatedly, the microneedles may retract into the initial position once the force is removed. The cradle attached to the blood collection device enables the users of the device to place digits consistently on top of the microneedles at an optimal position to provide consistent collection blood. This aspect is especially beneficial because the invention would provide consistent blood collection for users with varying digit sizes, especially for users of different age, and having digits with varying dimensions.

These devices may also have a compartment attached to the support to position a digit, such as a finger, thumb, or toe, within the compartment for piercing by the microneedles. The compartment is designed to guide engagement of the digit with the microneedles, and to hold the digit steady during the blood collection process. This is achieved by the compartment being a deformable material such as a polymer, which conforms to the shape of the digit when the digit is pressed into the compartment. The cradle may have holes aligned with the microneedles such that the microneedles can be deployed though the holes to pierce the skin of the finger or thumb held within the cradle. The microneedles may be mounted on a spring mechanism to actuate deployment of the microneedles. The actuation may be application of a force on the cradle by the finger or thumb. Relatedly, the microneedles may retract into the initial position once the force is removed. The compartment is especially advantageous to place the digits of the users, irrespective of their height, weight, age, digit length, or digit girth, to place the digits at an optimal position for consistent drawing of blood.

In a further aspect, the invention provides blood collection devices as described above, specifically a flexible embodiment of the support with an adhesive layer for securing to a patch of skin. The flexible support may be a stretchable and/or deformable material to conform to the shape of the area to which it is applied. The microneedles are arranged as a number of arrays within the support and activated by a spring mechanism to pierce the skin. In other embodiments, the array of microneedles is activated by pressing a button. The fill zones may be housed in a removable subportion of the support for easy release from the adhesive backing.

In related aspects, the invention provides a blood collection device with a flexible support as described above with a microneedle piercing apparatus or microneedle member that is releasably attached to the flexible support. The device includes a blood collection channel in fluid communication with the microchannels for directing the blood to the capillary microstructure of the device. The attachment mechanism allows the piercing apparatus to be removed from the device without damaging the blood collection channel or introducing contaminants into the blood collection process. In certain embodiments, releasing the piercing apparatus creates a vent to assist in drying the blood specimens collected. The microneedles may be deployed from the piercing apparatus or microneedle member via an actuation mechanism that retracts the microneedles once a predetermined quantity of blood has been collected. The flexible patch provides flexibility to collect blood samples from other parts of the body, including shoulder, legs (including thighs), forearms, buttocks, or back.

The components of the device are described in more detail below. In some embodiments, the support is configured to allow a user to insert a finger or other digit into the device and hold the digit in place until the fill zone receives a desired quantity of blood. In some embodiments of the device, the finger or other digit is held in place by a surface that conforms to the finger and guides a placement of the finger for piercing by the microneedles.

The device may also include a fill indicator that visibly shows when the fill zone has received a pre-determined quantity of blood. In some embodiments, the support is a substantially flat surface. For example, the support may be sized and configured to easily mail back to a testing laboratory. In other embodiments, the support is flexible to allow conformation to a surface to which it is applied.

Piercing Apparatus

The invention includes a novel finger puncture or piercing apparatus wherein the microneedles are integrated into the device to allow a user or consumer to puncture the skin of the user to collect blood. In particular, the device punctures skin on a digit, for example, a thumb, finger or toe. Embodiments of the piercing apparatus are designed to pierce a middle or ring finger. The apparatus may be designed to pierce a thumb. The piercing may be towards the tip and sides of the digit such that each user's skin is pierced in the same place, thus minimizing pain, standardizing the locus of the blood draw, and ensuring uniformity in the blood draw across several draws from the same user of the consistency of blood from separate users of the device. This consistency is important in conducting quantitative or qualitative comparisons on the blood collection device.

In other embodiments, the piercing apparatus is designed as part of a flexible blood collection device that may be adhered to a patch of skin, such as on a shoulder, thigh, or buttock. In this way, devices of the invention are not limited to blood collection from a digit, but may also be used to collect blood from other areas of the body.

In still other embodiments, the piercing apparatus is a standalone device that can be used in conjunction with the blood collection devices of the invention.

Piercing by the plurality of microneedles allows the blood to flow and to be directed to the blood collection well, the capillary microstructure, and to the fill zones within the device. Upon piercing, blood is wicked onto a material, such as filter paper, to facilitate movement of the blood to the blood collection well, the capillary microstructure and the fill zones. Finger prick apparatuses of the invention are capable of collecting from about 10 to about 300 μL of blood from a single prick. In certain embodiments, the devices of the invention are capable of collecting from about 130 to about 150 μL of blood in a single prick. The invention also provides that the dimensions or quantity of microneedles may be altered if a different quality of blood collection is required from the user for any diagnostic tests.

In aspects of the invention, the microneedle apparatus is integrated into the device as part of the blood collection well. In this regard, the invention provides for a device for collecting blood that includes a plurality of microneedles disposed on a support, such as described above. The blood collection device may have at least one fill zone disposed within the support, and capable of receiving and holding a quantity of blood. The blood collection device includes a capillary microstructure extending through at least a portion of the support and capable of conducting the blood from the microneedles into the fill zone. The blood collection device may also include a cradle positioning the digit of a user, such as a finger or a thumb, on a predetermined position on the device.

The apparatus allows blood to flow to the blood collection well, the capillary microstructure of the device, and the fill zones. In certain embodiments, the capillary microstructure of the blood collection device provides a separation mechanism that retains a cellular fraction of the blood at a predetermined location on the solid support and conducts plasma to the fill zone. The capillary microstructure directs blood to the separation media and conducts a cellular fraction of the blood to a predetermined location on the support and conducts plasma to the fill zone.

In some embodiments, the capillary microstructure directs blood to separation media or separation mechanism, which conducts a cellular fraction of the blood to a predetermined location on the support and conducts plasma to the fill zone. Separation may be active or may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

As integrated into the blood collection device, the plurality of microneedles direct blood to a collection well and the fill zones. Thus, the device may also include a capillary tube for extraction of blood from the piercing apparatus.

The user may apply a digit to the surface of the microneedles housed within the blood collection well such that the microneedles pierce the skin which allows the blood to begin flowing. As described in more detail below, the user may hold the digit in contact with the microneedles until a fill indicator indicates that a desired quantity of blood has been received within the fill zones. In some embodiments, the microneedles may retract into the device and are locked into the device after piercing the skin and the blood collection is complete. However, retraction of the needles may be tied to the fill indicator so that the needles will not retract until the metered quantity of blood is collected. This ensures that if the user lifts the digit before the quantity of blood has been received into the fill zones, the user may re-engage with the device to continue the blood collection process. Retraction of the needles back into the device enhances the safety of device and removes a biohazard element.

Compartments for Optimal Positioning of Digits on the Device

The invention provides blood collection devices comprising a compartment for positioning the digit of the user on a predetermined position on the device. In certain aspects of the invention, the compartment for positioning the finger is a cradle. In particular embodiments, the invention provides devices comprising a plurality of microneedles disposed on a support, at least one fill zone disposed within the support, wherein the fill zone is capable of receiving and holding a quantity of blood, a capillary microstructure, and a cradle positioning a finger or a thumb of the person at a predetermined position on the device. More specifically, the predetermined position on the device is on top of the microneedles. The compartment and/or cradle is especially advantageous to place the digits of the users, irrespective of their height, weight, age, digit length, or digit girth, to place the digits at an optimal position for consistent drawing of blood. In this application, the terms “compartment” and/or “cradle” refer to the appendage to the blood collection device.

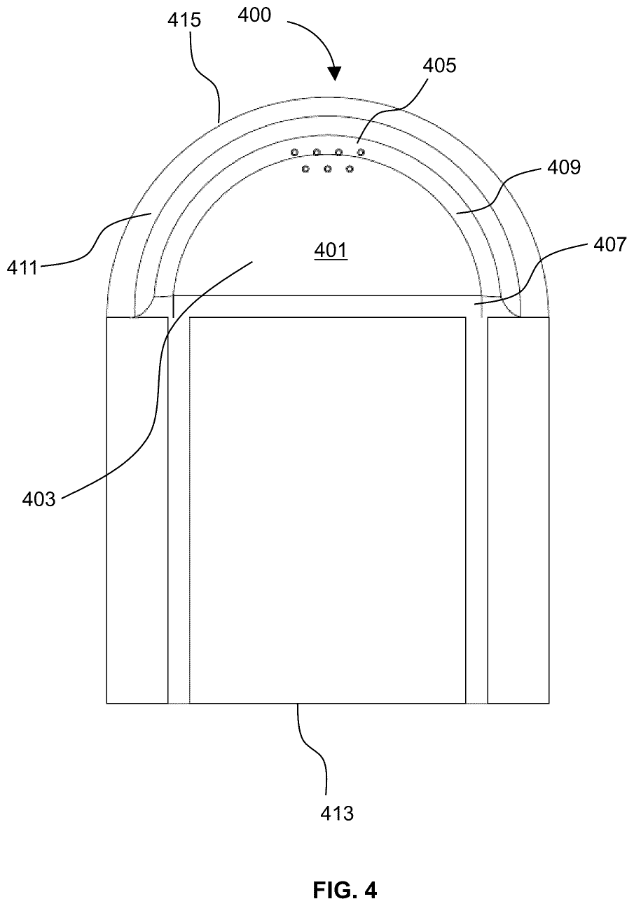

FIG. 4 illustrates a top view of one embodiment of a piercing apparatus 400 of the blood collection device utilizing an array of microneedles 405 positioned within the finger prick apparatus 401. The piercing apparatus may have a bottom 413 section and a top 415 section. In non-limiting embodiments, the finger prick apparatus 401 is shaped as a finger cradle 403 designed to hold a middle finger, ring finger, or thumb. The cradle 403 may be designed to guide engagement of the finger or thumb with the plurality of microneedles. For example, the apparatus may be formed to guide the tip of the digit to the microneedles 405 for piercing. In some embodiments, the cradle 403 may be raised in relation to the microneedles 405. For example, the apparatus may include a raised tip 407 that raises the cradle in relation to the microneedles 405. In non-limiting examples, the cradle may be raised 1/16 inch, ⅛ inch, ¼ inch, or ½ inch over the microneedles. In some embodiments, the length of the finger prick apparatus 401 is raised over the microneedles. In some embodiments the cradle is not raised over the microneedles. In some embodiments, an interior side 409 of the cradle is of a thickness that is less than an interior side of the rest of the apparatus. In some embodiments, an interior side 409 of the cradle is the same thickness as an interior side of the rest of the apparatus. In some embodiments, an interior side 409 of the cradle is of a thickness that is greater than an interior side of the rest of the apparatus.

In some embodiments, the piercing apparatus 400, which may be referred to as a lancet or microlancet includes a lip 411 around the apparatus. In non-limiting embodiments, the lip may have a thickness of 1/16 inch, ¼ inch, ½ inch, ¾ inch. The lip may be any thickness or width suitable for forming a guide and cradle for guiding, cradling, positioning, and/or holding, a finger for piercing.

The cradle 403 is designed for positioning a digit of a user on the device such that the digit is guided to engage with the microneedles of the device. The cradle is useful in providing consistent placement of the digit of the user at the same consistent position on the device, which leads to a uniform blood draw for the users of the device, independent of the dimensions of the digit of the user. Moreover, the consistent placement is also important to reduce the pain experienced by the user of the device. In particular, the placement may be such that the digit is guided on the device so that the nerve centers in the digits are positioned away from the microneedles, which results in reduced pain for the user. This is also beneficial in increasing the compliance of the users. The device also leads to a consistent amount of blood draw from repeated use of the device by the same user and/or use of device by different users having digits of different sizes.

It is important to have consistent blood draw across different users to reduce sample variability and have more consistent quantitative analysis. Another advantage of consistent blood draw is that a user would be assured that a requisite amount of sample is drawn from the finger resulting which avoids the user to submit the sample again due to lack of volume of blood required for any test.

The compartment and/or cradle may be prepared from a material such as plastic or elastic film, a deformable polymer, foam, or a combination of these materials. These compartments and/or cradles may have a tubular or conical shape, designed to guide the digit of the user to the top of microneedles. The cradle may also be comprised of a deformable or elastic material that deforms upon application of force by a digit of the user. In certain embodiments of the invention, the cradle may further include an attachment to hold the digit of the user on the predetermined position on the device for a duration in which the requisite amount of blood is collected. The attachment is particularly advantageous as it may reduce the instances where the required amount of blood is not collected from the user because the user may flinch upon contact with the microneedle and withdraw the digit prior to collection of the required amount of blood. The cradle may include a deformable material such as gel or foam to conform to the shape of the finger or thumb and hold the digit in place during the blood collection. In some embodiments, at least a portion of the cradle comprises material selected from a group consisting of: (i) plastic film, (ii) elastic film, (iii) a deformable polymer, (iv) foam, and (v) any combination thereof. For example, the cradle, or a portion thereof, may include a deformable polymer such as a viscoelastic silicone polymer. The cradle may conform to the shape of the finger or thumb upon application of pressure by the finger or thumb.

As mentioned above, the cradle is designed to guide a digit of the user to a predetermined position on the device, which is on top of the microneedles. Thus, the invention provides that the cradle has openings such that the digit of the user may contact the microneedles. These openings may be holes aligned with the microneedles such that the microneedles can be deployed though the holes to pierce the skin of the finger or thumb held within the cradle. These holes would be aligned such that when the digit of the user is optimally positioned, the microneedles go through the holes and contact the skin for blood collection. In another embodiment, the opening may be in a shape of a circular disk on top of the microneedles. This opening is important so that there is maximal contact between the digit and the microneedles resulting in optimal blood collection.

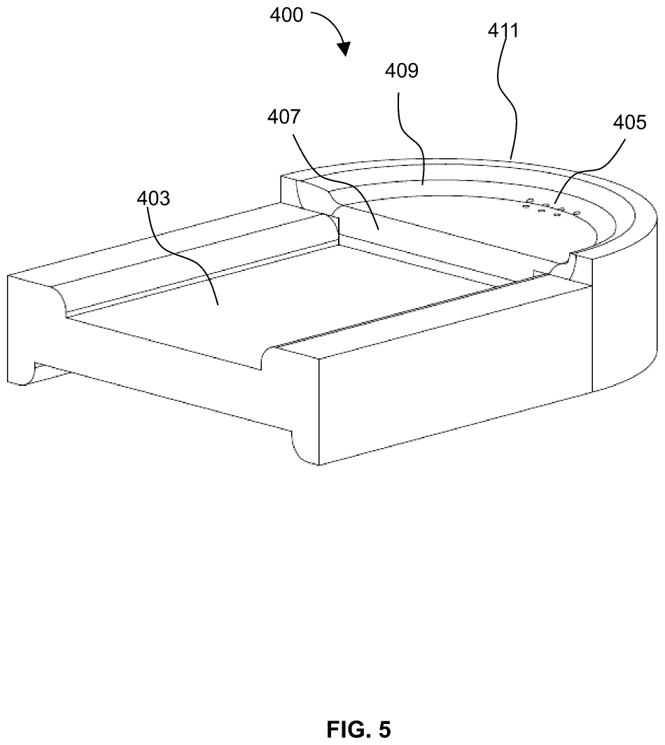

FIG. 5 illustrates a perspective view of a finger prick apparatus 400 according to one embodiment of the invention. As disclosed herein, the finger prick apparatus 400 may be a stand alone apparatus or the finger prick apparatus may be integrated into embodiments of the blood collection devices disclosed herein.

The finger prick device or microlancet device utilizes an array of microneedles 405 positioned within the finger prick apparatus. As noted, in non-limiting embodiments, the finger prick apparatus 400 is shaped as a finger cradle 403 designed to hold a middle finger, ring finger, thumb or other digit. The cradle 403 may be designed to guide engagement of the finger, thumb, or digit with the plurality of microneedles 405. As noted, in some embodiments, the area of finger prick apparatus containing the microneedles and/or the cradle 403 may be raised in relation to the microneedles 405. For example, the apparatus may include a raised tip 407 or platform that raises a portion or section of the cradle in relation to the microneedles 405. In non-limiting examples, the raised portion may be raised 1/16 inch, ⅛ inch, ¼ inch, or ½ inch over the microneedles 405 or in relation to the rest of the cradle 403. In some embodiments the cradle is not raised over the microneedles. In some embodiments, an interior side 409 of the cradle is of a thickness that is less than an interior side of the rest of the apparatus. In some embodiments, an interior side 409 of the cradle is the same thickness as an interior side of the rest of the apparatus. In some embodiments, an interior side 409 of the cradle is of a thickness that is greater than an interior side of the rest of the apparatus. The interior side 409 may be beveled or grooved to help guide, position, and hold the finger, thumb, or other digit in the finger prick apparatus

In some embodiments, the piercing apparatus 400, which may be referred to as a lancet or microlancet includes a lip 411 around the apparatus. In non-limiting embodiments, the lip may have a thickness of 1/16 inch, ¼ inch, ½ inch, ¾ inch. The lip may be any thickness or width suitable for forming a guide and cradle for guiding, cradling, positioning, and/or holding, a finger for piercing.

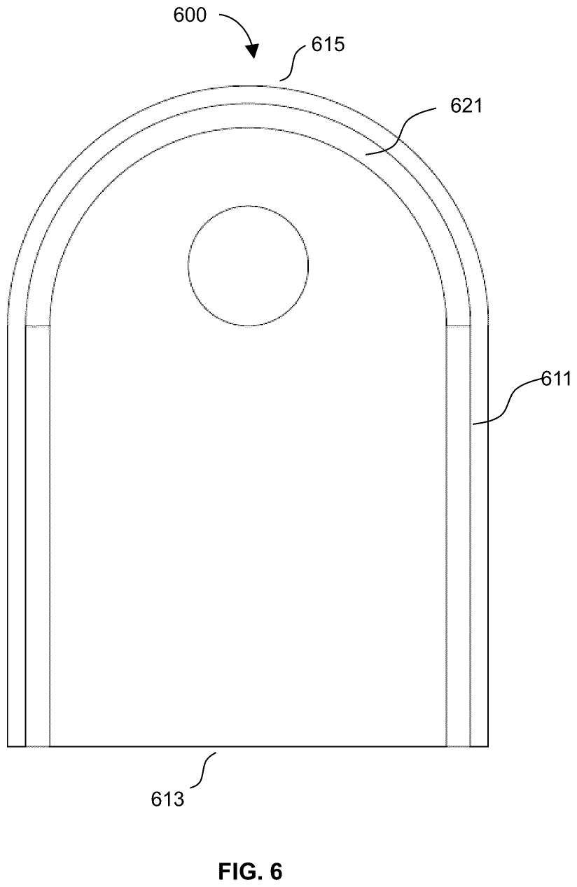

FIG. 6 illustrates a bottom view of one embodiment 600 of a finger prick apparatus according to the invention. The bottom of the finger prick apparatus may be substantially flat such that the apparatus 600 may be integrated into a center of a blood collection device, in some embodiments. The apparatus may be shaped as a cradle as described above and integrated into the blood collection device. As shown, the apparatus 600 may have a top edge 615 and a bottom edge 613 for orientation. The bottom may be of a flat configuration for insertion of a thumb. The apparatus may have hollow or solid microneedles. The cradle may be shaped to conform to the digit of the user, particularly it may be shaped to form to and guide a user's thumb into the apparatus to make contact with the microneedles. The cradle may include a guide that forms the perimeter of the cradle except for the bottom of the cradle which may be open and flat for the digit to slide into. For example, the apparatus 600 may include a lip 611 to guide and hold a digit, for example a thumb. In some embodiments, the lip 611 has a thickness from about 0.0625 inch to about 0.5 inch. In other embodiments, the lip 611 has a thickness of about 0.25 inches. In some embodiments, the lip 611 has a width from about 0.0625 inch to about 0.5 inch. In other embodiments, the lip 611 has a width of about 0.25 inches.

The apparatus 600 may include a groove 621 around the top of the apparatus. The groove may be used to help position the digit within the apparatus.

The apparatus may utilize an array of spring-loaded microneedles positioned within the cradle of the finger prick apparatus. The microneedles may be hollow. The microneedles may be solid. The microneedles may be a combination of both solid and hollow. The bottom surface may include a texture to help keep the digit positioned within the apparatus during the blood collection procedure.

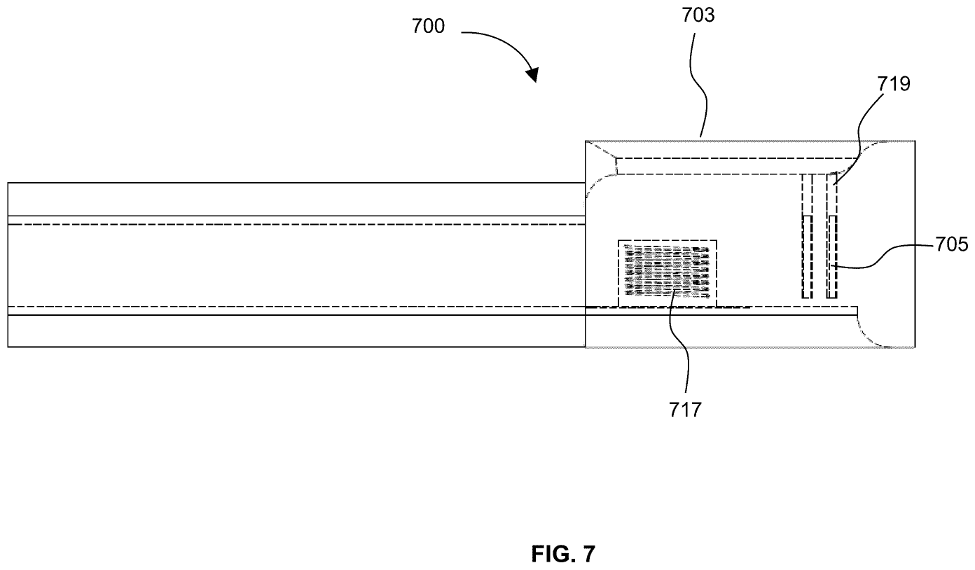

FIG. 7 illustrates a side view of a further finger piercing apparatus 700 that utilizes an array of spring-loaded microneedles 705 positioned within the cradle of the finger prick apparatus. The microneedles 705 may be solid or hollow or a combination of solid and hollow. The microneedles 705 may be mounted on a spring mechanism 717 to actuate deployment of the microneedles 705. The actuation may be application of a force on the cradle 703 by the finger or thumb. This is advantageous by avoiding the accidental deployment of microneedles so that they are deployed only when the digit of the user is optimally positioned for blood collection. The actuation mechanism may be a spring-loaded mechanism 717 which is actuated by the application of force. For example, the spring-loaded mechanism may be actuated/released when a set force is loaded. Further, when the force is released, the spring-loaded mechanism may lock back in place. The locking mechanism may have a time delay after being releases such that the actuation mechanism may be re-engaged. The actuation may also happen by pressing a button. Relatedly, the microneedles retract into the initial position once the force is removed and/or the blood collection is complete. The retraction of the microneedles after the sample collection ensures there is no biohazard in shipping the device after the blood collection is complete.

In some embodiments, the plurality of microneedles are mounted on a spring mechanism. Thus, the apparatus uses a plurality of spring-loaded microneedles that are deployed when the force of the finger in the cradle is exerted on the spring mechanism. the spring mechanism is actuated upon application of a predetermined amount of force on the cradle by the finger or thumb, resulting in engagement of the microneedles with the finger or the thumb of the person. In some embodiments, once the application of force is removed, the plurality of microneedles retract to an initial position prior to actuation. Retraction of the needles back into the device enhances the safety of device and removes a biohazard element. Moreover, this retraction also provides the option of sending the collected sample for analysis.

The cradle 703 may also include holes 719 aligned with the plurality of microneedles 705 which allows the plurality of the microneedles 705 to protrude therethrough and puncture the finger held within the device 700. Thus the microneedles 505 may be deployed through the holes 719 to pierce the skin and may be retracted back therefrom. In some embodiments, the cradle 703 has a thickness from about 0.0625 inch to about 0.5 inch. In other embodiments, the cradle has a thickness of about 0.25 inches. This allows for the piercing apparatus or microlancet to be integrated into the blood collection device such that the blood collection device is substantially flat. The width of the cradle may be from about 0.01 inch to about 1 inch, or preferably, it is about 0.5 inch. In some embodiments, the piercing apparatus may include a lip for positioning a thumb within the device.

The microneedles may be positioned to pierce the digit on the top and side and away from pain centers. The plurality of microneedles may contain an analgesic coating and/or an anticoagulant coating. The apparatus holds the finger steady during piercing and blood collection such that the blood collection is a uniform quantity and quality. In some embodiments, the cradle allows engagement with the finger or thumb in place for a preset duration of time resulting in collection of sufficient quantity of blood. This can be done via the integrated fill indicator, as described in more detail below. The fill indicator may visibly show when the fill zone has received a pre-determined quantity of blood.

As described herein, the piercing apparatus, containing the microneedle, may be a standalone apparatus. As a standalone apparatus, the piercing apparatus allows the user to simply pierce the skin of a digit and to then drop the blood into the blood collection well of the device. Specifically, the piercing apparatus, containing the microneedles, allows a user to puncture their finger, such as a middle or ring finger, and apply blood from this puncture to the blood collection well of the blood collection device. The piercing may yield from about 10 to about 300 μL of blood. In certain embodiments, the piercing yields from about 130 to about 150 of blood.

The standalone piercing apparatus may be a compartment. The compartment may be shaped, for example, like a thimble or covered tube, such that a user can insert a digit into the compartment to contact the plurality of microneedles. In this way, the user does not see the blood drawn from the piercing by the microneedles. The compartment may include a locking mechanism that retracts the microneedles into the locking mechanism to protect the microneedles after use so as not to be considered hazardous waste. Thus, retraction of the needles back into the device enhances the safety of device and removes a biohazard element.

The piercing apparatus or device may be a standalone cradle with solid or hollow microneedles such as the cradle integrated into the blood collection device and described above. The standalone device may have a flat underside so that the device may be stably placed on a surface for inserting a finger or thumb. The cradle may have a deformable base that conforms to the inserted finger or thumb. The deformable material may be a gel or foam to conform to the shape of the finger or thumb and hold the digit in place during the blood collection. In some embodiments, at least a portion of the cradle comprises material selected from a group consisting of: (i) plastic film, (ii) elastic film, (iii) a deformable polymer, (iv) foam, and (v) any combination thereof. For example, the cradle, or a portion thereof, may include a deformable polymer such as a viscoelastic silicone polymer. The cradle may conform to the shape of the finger or thumb upon application of pressure by the finger or thumb.

For example, a user may place the piercing apparatus on a table and place a digit into the apparatus such that the deformable base of the apparatus holds the digit in place. As described below, the microneedles may be attached to a spring-loaded mechanism that is activated by force applied to the mechanism to deploy the microneedles and pierce the digit. Alternatively and/or additionally, the microneedles may be deployed by contact with the digit inserted into the apparatus.

The apparatus may have an array of microneedles used to puncture the digit such that the user can then place blood into the blood collection device.

In other embodiments, the piercing apparatus is a blood lancet using an array of microneedles. The blood lancet may be similar to a scalpel style lancet but with an array of microneedles for puncturing the skin. The microneedles may be spring-loaded to deploy for puncture and to automatically retract after piercing the skin. Retraction of the needles back into the device enhances the safety of device and removes a biohazard element. In some embodiments, the blood lancet yields from about 10 to about 300 μL of blood. In certain embodiments, the piercing yields about 130 to about 150 μL of blood.

Aspects of the invention provide for a device for collection of a blood sample comprising a plurality of microneedles disposed on a support, and a compartment attached to the support. In this aspect, the compartment is designed to position a digit at a predetermined position on the apparatus. For example, the digit may be a finger, thumb, or toe of an individual. This compartment is advantageous to provide consistent placement of the digit on the device. Moreover, the compartment is designed to provide consistent placement to digits of varying dimensions. This results in consistent blood collection across users of different age, height, weight, and digit dimensions. The device may have at least one fill zone disposed within the support, capable of receiving and retaining a quantity of blood. The device may also include a capillary microstructure extending through at least a portion of the support and capable of conducting the blood from the plurality of microneedles into the fill zone.

In some embodiments, the compartment is designed to guide engagement of the digit with the plurality of microneedles. For example, the compartment may comprise a deformable material that guides the digit into the compartment and conforms to the shape of the digit to hold the digit in place for piercing by the microneedles. The deformable material may be a gel or foam to conform to the shape of the digit and hold the digit in place during the blood collection. In the deformable material is selected from a group consisting of: (i) plastic film, (ii) elastic film, (iv) non-woven materials, (v) a deformable polymer, foam, and (vi) any combination thereof. The deformable material may be a polymer, such as a flexible putty or shape-memory polymer. For example, the deformable material may be a polymer such as a viscoelastic silicone polymer. The deformable material conforms to the shape of the digit upon application of pressure by the digit.

In other embodiments, the compartment may be cradle-shaped as described above. The compartment may have a housing designed to guide the digit at the predetermined position on the device. The compartment may comprise an elastic film, such as linear low-density polyethylene (LLDPE).

The compartment may include one or more openings, aligned with the plurality of microneedles, allowing the plurality of the microneedles to protrude therethrough to the digit. The microneedles may be mounted on a spring mechanism that is actuated by application of a predetermined amount of force on the compartment by the digit, resulting in engagement of the microneedles with the digit. When application of the force of the digit is removed the plurality of microneedles may retract to an initial position prior to the actuation. Thus, the microneedles are contained within the device and no longer exposed. Retraction of the needles back into the device enhances the safety of device and removes a biohazard element.

The compartment may allow engagement with the digit for a preset duration of time resulting in collection of sufficient quantity of blood. The preset duration of time is calibrated to allow for a sufficient quantity of blood to be collected. The sufficient quantity of blood collected may be determined by a fill indicator integrated into the device that visibly shows when the fill zone has received a pre-determined quantity of blood. The plurality of microneedles may contain an analgesic coating, for example any topically active analgesic agent, including, but not limited to benzocaine, butamben, dibucaine, lidocaine, oxybuprocaine, pramoxine, proxymetacaine (proparacaine), and tetracaine, and/or an anticoagulant, for example, coumarins, heparins, or any other factor Xa inhibitor. The anticoagulants facilitate more consistent blood flow from the puncture by temporarily reducing the blood clotting. The analgesics are beneficial in alleviation of pain and thus increasing compliance for use of the blood collection device.

The blood collection device includes a capillary microstructure that may provide a separation mechanism that retains a cellular fraction of the blood at a predetermined location on the solid support and conducts plasma to the fill zone.

In some embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

Flexible Device for Adhering to Skin Surfaces

In certain aspects of the invention, the blood collection device includes flexible support such that the device is capable of deploying on other parts of a user's body such as a shoulder, thigh, or buttocks. The flexible support may include an adhesive backing for applying to the body to keep the device in place for the blood collection procedure. The invention provides devices for an easy-to-use blood collection experience. Specifically, the invention provides blood collection devices that allows a user to collect an accurately metered quantity of blood stored within the device for use by diagnostic testing laboratory. Thus, devices of the invention allow for remote blood collection which provides access to necessary blood testing in areas lacking local and easily accessible laboratory services.

The invention provides a blood collection device that can be applied as a patch on the body. These devices include a plurality of microneedles disposed on a flexible support. The flexible support has a first side for contacting skin comprising a microneedle assembly comprising a plurality of microneedles and a second side opposite to the first side. The microneedle assembly is designed to contact and pierce the skin to initiate the blood collection. The microneedle assembly may be releasably attached to the flexible support. The devices of the invention further comprise a blood connection channel, wherein the blood connection channel is in fluid communication with the base of at least one microneedle in the microneedle assembly and an attachment mechanism for manually releasing the plurality of microneedles without damaging the blood connection channel.

The blood collection devices are particularly advantageous because they can be applied to several spots on the users' body to collect the optimal amount of blood. For example, the device of the invention may be applied on the shoulder, biceps, back, hip, buttocks, or leg of the user. It is also beneficial to have a releasable microneedle assembly in the patch because the microneedle assembly may be removed from the device prior to mailing the device for analysis. The removal of microneedle assembly from the device removes the biohazard and allows for safe transfer of the device comprising the collected blood sample for further analysis.

The blood collection devices are particularly advantageous because they can be applied to several spots on the users' body to collect the optimal amount of blood. For example, the device of the invention may be applied on the shoulder, biceps, back, hip, buttocks, or leg of the user. It is also beneficial to have a releasable microneedle assembly in the patch because the microneedle assembly may be removed from the device prior to mailing the device for analysis. Beneficially, the devices of the invention do not rely on gravity to collect blood from the user. This is important because it can be applied at various locations on body of user in a configuration where the blood collection mechanism does not rely on gravity to collect the blood. For example, it can be used on thigh of user, where the user of the device would be able to observe the collection of the blood from the device. The aspect of user being able to monitor the patch provides several advantages. In particular, the user may be able to analyze if there are any reactions to the device on the skin surrounding the area where the device is applied. The user would also be able to monitor the amount of blood being collected in the device. The removal of microneedle assembly from the device removes the biohazard and allows for safe transfer of the device comprising the collected blood sample for further analysis.

The flexible support of the blood collection device housing the plurality of microneedles is designed to conform to the shape of the body where the blood collection is applied as a patch. The flexible support conforms to the shape of the body such that when the plurality of microneedles housed in the flexible patch may contact the skin. The flexible support may comprise a polymer with high elasticity and deformability. The support may vary in thickness to meet the needs of the device, such as about 5000 micrometers or less, in some embodiments from about 1000 to about 2000 micrometers, from about 1 to about 500 micrometers, and in some embodiments, from about 10 to about 200 micrometers. The flexible support may include at least one of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, Gore-Tex, plastic, plastic components, polymer, biopolymer, woven material, non-woven material, and natural material. The flexible support may also comprise a polyurethane-based film. In some embodiments, the flexible support has a shape comprising at least one of a circle, oval, ellipse, square, rectangle, triangle, diamond, butterfly, and hourglass. The invention further provides that the flexible support is larger than the area of microneedle assembly. Thus, there is an area surrounding the microneedle assembly that contains backing with no microneedles. This design makes it easier to apply the device. It also reduces the risk of being unintentional contact of the microneedle assembly with the skin either during application or removal. The unintentional contact of the microneedle assembly with the skin can result in bleeding or infection. This is especially important if another person applies or removes the device as this will reduce the risk of transmitting infections between them. The microneedle assembly may be placed in the center of the backing or off center.

The device of the invention may further comprise an adhesive layer that can help facilitate the attachment of the patch to a user's skin during use. The adhesive layer typically employs an adhesive coated onto a backing material. The backing may be made of a material that is substantially impermeable to blood, such as polymers, metal foils, etc. Suitable polymers may include, for instance, polyethylene terephthalate, polyvinylchloride, polyethylene, polypropylene, polycarbonate, polyester, and so forth. The adhesive may be a pressure-sensitive adhesive. Suitable adhesives may include, for instance, solvent-based acrylic adhesives, solvent-based rubber adhesives, silicone adhesives, etc.

The devices of the invention include the use of microneedle assembly comprising a plurality of microneedles as the piercing mechanism for the blood draw. The microneedles can be hollow and/or solid. Additionally, the microneedles may be coated with an analgesic to reduce the pain of the piercing, and/or an anticoagulant to minimize clotting. The microneedles may also be in a piercing apparatus that is attached to the surface of the blood collection device, for example shaped as a button that can be removed upon completion of the blood collection process. For example, the compartment may be attached via a snap or latch, or by a tab. In some configurations, the microneedles are actuated to pierce the skin by pressing on the button. The microneedles then retract back into the compartment once blood collection is complete. The compartment can then be removed from the device and thrown away. For example, the compartment may be removed by unlatching, unsnapping, or twisting off the compartment. The microneedles are arranged as a number of arrays within the support.

The microneedle assembly may be activated by a spring mechanism to pierce the skin. The spring may be activated by application of pressure after application of the patch on the skin of the user. In other embodiments, the spring is actuated by pressing a button, which results in activation of the microneedle assembly. The spring may include a battery spring or coil spring or another spring or component, as long as it can store energy. In other embodiments, spring may be an air spring, an elastomer, a foam, another fluid spring, a gas spring, another highly compressible material(s), a leaf spring, a sponge, or another member that stores energy (e.g., mechanical or potential energy). The invention further provides that the microneedle assembly may retract in the flexible backing after the blood collection.

The blood collection channels of the inventions further comprise a capillary structure to transfer the blood from the plurality of microneedles to the fill zones, wherein each of the fill zones receives and holds a predetermined volume of the blood. Blood is collected in the blood collection well by the integrated microneedles or from the microneedle piercing apparatus. From the blood collection well, the blood enters the capillary microstructure to separate the blood components and fill the respective fill zones. The capillary microstructure of the devices is contained within layers of the support and defined by barriers, such as hydrophobic wax barriers. The hydrophobic wax barriers also define the blood collection well or sample addition zone, the fill zones, and the fill indicator. The layers of the support allow for the separation of the components the blood collection into discrete fill zones. For example, layers of the device may include a top and bottom, a laminate layer on a top portion of the device for sealing, layers of polyester mesh, clear polyethylene terephthalate (PET) mylar membranes, polyester membranes, clear PETG membranes, asymmetrical polysulfone membranes, double-sided adhesive to affix the membranes, and a cellulose or chromatography paper layer or cut out. The blood collection well may include a substrate that is a hydrophilic, porous media for receiving blood. The invention advantageously provides that the fill zones are housed in a removable subportion of the device such that the fill zones may be removed from the remaining device after the collection of the blood sample. The removal of fill zones comprising the collected blood is beneficial because of the ease of shipping the collected blood sample for further analysis. Preferably, the removable subportion of the device comprising the fill zone includes an identification tag for identifying the patient. The identification tag may be a QR code, bar code, RFID tag, or a label including the patient's information. The fill zones may be housed in a removable subportion of the support for easy release from the adhesive backing.

The device of the invention may further include a fill indicator. The fill indicator that may be a color-based fill indicator which changes color after collection of a predetermined quantity of blood in the blood collection wells. In other aspects, the fill indicator may be a visible mark, such as a tick mark, which becomes visible or changes color upon collection of a predetermined quantity of blood in the blood collection wells.

The blood collection devices of the invention optionally include an antiseptic and analgesic. The blood collection devices of the invention include microneedles as the piercing mechanism, a blood collection well, a capillary microstructure that separates plasma from whole blood and directs each to separate fill zones within the device, and a fill indicator. The fill indicator may be metered or non-metered. The device may be housed on a solid support or as a flexible and deformable support designed for placing on a patch of skin on the body. The support includes various layers to achieve the functionality of the devices, and hydrophobic wax barriers to define the different features of the devices.

Certain devices of the invention include integrated microneedles to pierce skin and direct a metered quantity of blood, either as whole blood or plasma, to fill zones within the device. The microneedles of the devices are positioned away from pain centers for a painless or reduced-pain puncture as compared to traditional lancets. The microneedles are aligned such that blood draw and collection are standardized across samples and users, thus eliminating variability in quantity and quality of the blood draw. A blood collection well, a capillary microstructure, and a fill indicator, also integrated into the devices, acting in conjunction with the microneedles, ensures that blood flowing from the puncture area is not visible to the user. Thus, the invention provides for a pleasant, uniform puncture that achieves a metered volume of blood, and eliminates operator error and sample contamination issues.

In preferred embodiments, devices of the invention allow the collection, separation, and storage of sufficient quantities of different components of blood, such as white blood cells, red blood cells, platelets, and plasma, in a single device. For example, devices of the invention store the collected blood as whole blood, or the devices are used to separate plasma from whole blood and store each as separate samples on the same device. This allows for a sufficient quantity of blood for multiple diagnostic testing. This is especially necessary for remote locations in areas with limited testing facilities. For example, using blood and plasma collected and separated in a single device, plasma can be used to test for a virus, and then, using the results of the plasma testing, whole blood can be tested to determine the prescription for or treatment of the virus. Devices of the invention also avoid or minimize hemolysis and provide enhanced blood specimen stability for collection, storage, maintenance, and transport. Devices of the invention provide improved blood specimen stability over a range of ambient conditions for extended durations. Blood samples are also stabilized over varying environmental conditions, including conditions with high ambient humidity, and a varying range of pressures. Again, this is important for remote locations away from local laboratory testing facilities. Thus, the invention integrates all aspects of the blood collection experience into a lightweight and flexible support structure convenient for mailing back to a laboratory for testing. Upon reconstitution, blood samples collected by, and stored within, the device are comparable to samples obtained by venous blood draw or other method of drawing blood for analysis.

In certain embodiments, the devices of the invention and/or the piercing apparatuses provide for retracting the microneedles back into the piercing apparatus or the device itself after blood collection is complete. In this way, the biohazard waste element is removed so the device can be mailed to the diagnostic testing laboratory.

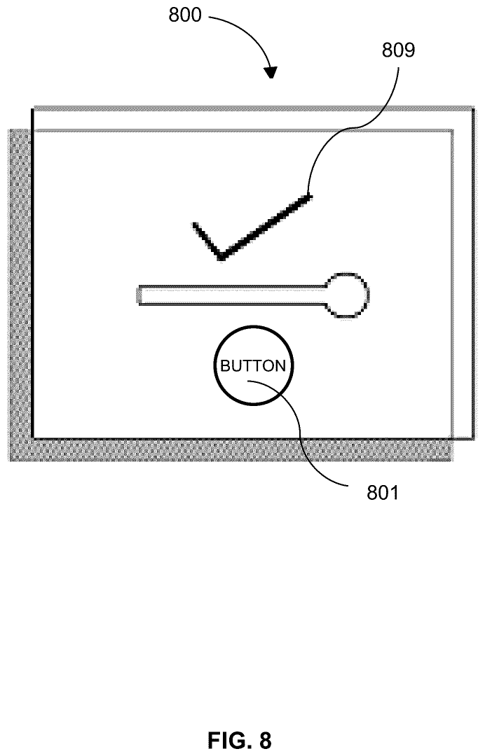

FIG. 8 illustrates a top view of one embodiment of a flexible blood collection device 800, designed to adhere to a user's skin. In this embodiment, the device includes a piercing apparatus 801. The blood collection device may be housed on a flexible support capable of being molded to a surface of the user's body, such as a shoulder. The blood collection device may include a removable piercing apparatus 801, for example shaped as a button, containing the plurality of microneedles. The blood collection device may include a fill indicator 809.

Aspects of the invention provide for a blood collection device made of a flexible material having a first side for contacting skin and an opposed second side. The blood collection device may include a microneedle member, or piercing apparatus, releasably attached to the material. The microneedle member may include at least one microneedle positioned to pierce skin when the skin is in contact with the first side. As with other devices of the invention, the blood collection device may include a blood collection channel within the material, wherein a proximal portion of the blood collection channel is in fluid communication with a base of the at least one microneedle.

The blood collection device may include an attachment mechanism for manually releasing the microneedle member from the material without damaging the blood collection channel. For example, the attachment mechanism may be configured such that the microneedle member may be twisted off to release it from the material.

The flexible support may be a stretchable and/or deformable material to conform to a shape of an area to which the device is applied. The support may be, for example, plastic, cellulose, cardstock, cover stock, pasteboard, paperboard, fiber board, rubber, urethane, or silicone. The support may also include a flexible mesh fabric or woven material for flexibility in the application of the device. The support may be one or more of materials that provide the balance of rigidity, flexibility, weight, and durability to house the components of the device. For example, the support may be a woven fabric, plastic, such as PVC, polyethylene or poly urethane, or latex. In some embodiments, the flexible material comprises a stretchable and/or deformable material to conform to a shape of an area to which the device is applied.

The support may include one or more layers, for example, a laminate layer on a top portion of the device for sealing, layers of polyester mesh, clear PET mylar membranes, polyester membranes, clear PETG membranes, asymmetrical polysulfone membranes, double-sided adhesive to affix the membranes, and a cellulose or chromatography paper layer or cut out. The first side, or underside, of the blood collection device may include an adhesive layer to secure the device to the skin. The adhesive layer may be covered with a removable adhesive backing such that the removable adhesive backing is removed prior to applying the device to skin. The adhesive may be, for example, an acrylate or vinyl resin, safe for adhesion of the device to skin.

As detailed above, the support may be a patterned dried blood spot support that includes one or more barriers disposed on the one or more layers. The barriers may be hydrophobic wax barriers. For example, the wax barriers may be formed by wax printing using a double-sided wax-transfer method to pattern the support. For example, the top and bottom designs may be printed onto laminate sheets, using a wax printer, for example a Xerox ColorQube 8580 wax printer. The layers, for example, the chromatography layer, may be aligned with the top and bottom designs. Alignment can be achieved by a clamp or alignment jig. The wax from the laminate sheets may be transferred to the chromatography paper using a heat press, for example a Promo Heat CS-15, to form the hydrophobic wax barriers. The hydrophobic wax barriers are patterned to define the different parts of the device.

In certain embodiments, the microneedles are housed in a removable microneedle member, or piercing apparatus, disposed on the flexible support. The piercing apparatus may be a plastic case that removably attaches to the support of the blood collection device. For example, the piercing apparatus may be shaped as a button 801 housed on the top side of the flexible support of the blood collection device 800.

In certain embodiments, the microneedles are housed in a removable microneedle member, or piercing apparatus, disposed on the flexible support. The piercing apparatus may be a plastic case that removably attaches to the support of the blood collection device. For example, the piercing apparatus may be shaped as a button 801 housed on the top side of the flexible support of the blood collection device 800. In some embodiments, the button may be spring-loaded such that when the blood collection device is placed on the skin of a use, the button is pushed and the microneedles are engaged. When the fill indicator In some embodiments of the device, the blood collection channel comprises a capillary structure that carries blood away from the at least one microneedle. Blood may be wicked by the microneedles to the blood collection channel and into the capillary microstructure to the fill zones. In non-limiting examples, blood may be transported via capillary action. The blood collection devices are particularly advantageous because they can be applied to several spots on the users' body to collect the optimal amount of blood. For example, the device of the invention may be applied on the shoulder, biceps, back, hip, buttocks, or leg of the user. It is also beneficial to have a releasable microneedle assembly in the patch because the microneedle assembly may be removed from the device prior to mailing the device for analysis. Beneficially, the devices of the invention do not rely on gravity to collect blood from the user. This is important because it can be applied at various locations on body of user in a configuration where the blood collection mechanism does not rely on gravity to collect the blood. For example, it can be used on thigh of user, where the user of the device would be able to observe the collection of the blood from the device. The aspect of user being able to monitor the patch provides several advantages. In particular, the user may be able to analyze if there are any reactions to the device on the skin surrounding the area where the device is applied. The user would also be able to monitor the amount of blood being collected in the device. The removal of microneedle assembly from the device removes the biohazard and allows for safe transfer of the device comprising the collected blood sample for further analysis.

The distal portion of the blood collection channel may include a blood fill zone of a predetermined volume. For example, the fill zone may be at least 10 μL.

The blood collection device may include a fill indicator to indicate an amount of blood being collected in the fill zone. For example, the microneedle member may display an exposed base visible on the opposed second side, with a fill indicator visible on the exposed base. For example, the fill indicator may be a color change indicator integrated into the adhesive portion or strip of the device. The adhesive strip may be applied to the body of the user for a period of time until a portion of the strip, defined as the fill indicator, changes color. For example, the top of the adhesive strip may comprise the fill indicator such that when the metered volume of blood has been achieved the top of the adhesive strip changes color.

In certain embodiments, the microneedle member is attached via an attachment mechanism. The attachment mechanism may be a base structure fixedly attached to the flexible member such that the microneedle member is releasably attached to the base structure by a releasable snap-fit or threaded attachment.

The microneedles housed within the microneedle member may be hollow or solid, or a combination of both. In some embodiments, the microneedle member, or piercing apparatus, is attached to an actuation mechanism such that the actuation mechanism is actuated for the microneedle member to pierce skin. For example, the microneedles may be connected to a spring mechanism within the microneedle member. Thus, activation of the spring mechanism inserts the plurality of microneedles in the skin of the subject. The plurality of microneedles housed within the microneedle member, or piercing apparatus, may be spring-loaded such that application of force to the outside of the microneedle member deploys the microneedles. For example, for embodiments in which the microneedle member is shaped as a button, the user pushing on the button is a force sufficient to actuate the microneedles to pierce the skin.

In other embodiments, the spring mechanism may be activated upon application of the device on the skin of the subject. The microneedles may then pierce the skin of the user and begin the blood collection process. The microneedle member, or piercing apparatus, may include a momentary switch or release that allows the microneedles to be deployed upon actuation by the user. The momentary switch may actuate the microneedles and hold them in a piercing position without the continued application of force. In this embodiment, a second application of force on the microneedle member, once a metered quantity of blood has been collected, retracts the microneedles back into the microneedle member. In this way, in embodiments, the activation and retraction of the microneedles of the blood collection device occur with a simple push of a button—once to deploy the microneedles, and once to retract the microneedles.

The microneedles may stay engaged with the skin of the user and the blood collection device until the metered quantity of blood has been received by the fill zones as indicated by the fill indicator. In this embodiment, completion of collection of blood, as indicated by the fill zone, causes the microneedles to retract into the piercing apparatus such that the microneedles are not exposed. Retraction of the needles back into the microneedle member enhances the safety of device and removes a biohazard element.

The microneedle member, or piercing apparatus, may be removed from the solid support and discarded with the microneedles contained within the apparatus. Removal of the microneedle member may create a vent in the blood collection device that facilitates drying of the blood collected in the fill zones.

In still further embodiments, the blood fill zone is removable from the flexible material. The blood fill zone may be housed in a covering that includes a barcode to identify the user/patient. For example, embodiments of the invention may include a plurality of fill zones on the device, such that the fill zones together are removed from the flexible material as one piece identified by the barcode and suitable for mailing to the testing laboratory.

In aspects of the invention, the blood collection device comprises a plurality of microneedles disposed on a flexible support, at least one fill zone capable of receiving and holding a quantity of blood, a capillary microstructure extending through at least a portion of the support and capable of conducting the blood from the microneedles into the fill zone, and an adhesive pad comprising an adhesive layer for securing the device to skin.

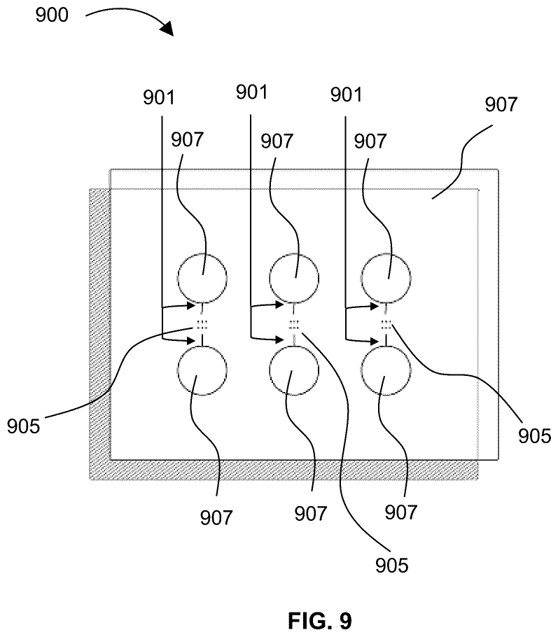

FIG. 9 illustrates a bottom view of one embodiment of a flexible blood collection device 900 designed to adhere to a user's skin, showing the fill zones 907 for separation of blood and plasma, integrated microneedles 905, and channels 901 to the fill zones 907.

The flexible support may a stretchable and/or deformable material to conform to a shape of an area to which the device is applied. The support may be, for example, plastic, cellulose, cardstock, cover stock, pasteboard, paperboard, fiber board, rubber, urethane, or silicone. The support may also include a flexible mesh fabric or woven material for flexibility in the application of the device. The support may be one or more of materials that provide the balance of rigidity, flexibility, weight, and durability to house the components of the device. For example, the support may be a woven fabric, plastic, such as PVC, polyethylene or poly urethane, or latex. In some embodiments, the flexible material comprises a stretchable and/or deformable material to conform to a shape of an area to which the device is applied.

The flexible support may comprise one or more layers for example, a laminate layer on a top portion of the device for sealing, layers of polyester mesh, clear PET mylar membranes, polyester membranes, clear PETG membranes, asymmetrical polysulfone membranes, double-sided adhesive to affix the membranes, and a cellulose or chromatography paper layer or cut out. In some embodiments, the layers may comprise one or more layers of filter paper 923. The first side, or underside, of the blood collection device may include an adhesive layer 925 to secure the device to the skin. The adhesive layer may be covered with a removable adhesive backing such that the removable adhesive backing is removed prior to applying the device to skin. The adhesive may be, for example, an acrylate or vinyl resin, safe for adhesion of the device to skin.

As detailed above, the support may be a patterned dried blood spot support that includes one or more barriers disposed on the one or more layers. The barriers may be hydrophobic wax barriers. For example, the wax barriers may be formed by wax printing using a double-sided wax-transfer method to pattern the support. For example, the top and bottom designs may be printed onto laminate sheets, using a wax printer, for example a Xerox ColorQube 8580 wax printer. The layers, for example the chromatography paper layer, may be aligned with the top and bottom designs. Alignment can be achieved by a clamp or alignment jig. The wax from the laminate sheets may be transferred to the chromatography paper using a heat press, for example a Promo Heat CS-15, to form the hydrophobic wax barriers. The hydrophobic wax barriers are patterned to define the different parts of the device.

The capillary microstructure may comprise channels 901. The capillary microstructure may include a separation mechanism in the channels that retains a cellular fraction of the blood at a predetermined location on the solid support and conducts plasma to the fill zone. The separation may be achieved by virtue of the capillary structure of the device. Thus, the microstructure is capable of separating plasma and whole blood fractions into separate fill zones on the device.

In embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices

In some embodiments of the device, the blood collection channel comprises a capillary structure that carries blood away from the at least one microneedle. In a non-limiting example, blood may be carried away from the at least one microneedle through capillary action. Blood may be wicked by the microneedles to the blood collection channel and into the capillary microstructure to the fill zones. The distal portion of the blood collection channel may include a blood fill zone of a predetermined volume. For example, the fill zone may be at least 10 μL.

The microneedles housed within the device may be hollow or solid, or a combination of both. In some embodiments, the microneedles are hollow. The device may include a plurality of microneedle arrays spaced throughout the device. The microneedles may be coated with an analgesic such as lidocaine, and/or an anticoagulant such as heparin.

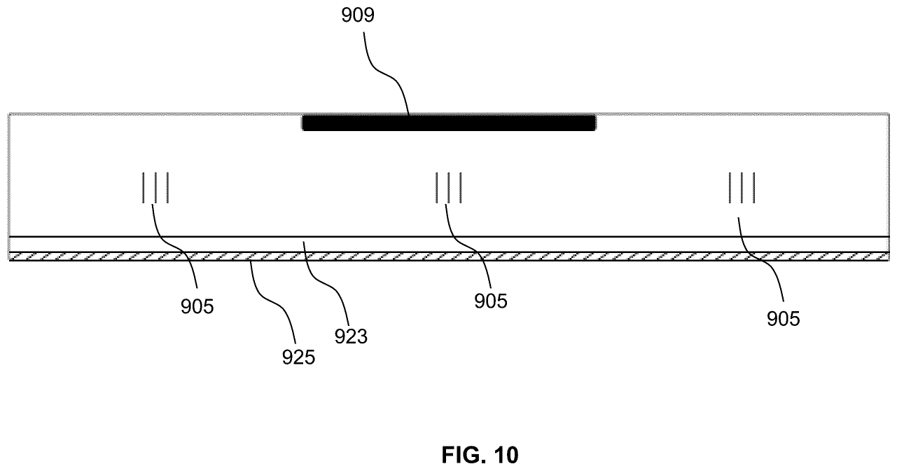

FIG. 10 illustrates a side view of the device 900 designed to adhere to a user's body. The fill indicator 909 may be on the top of the device, as a window visible to the user. As noted above, the support may comprise a plurality of layers, for example a layer of filter or chromatography paper. The support may be a flexible plastic case with the microneedles 905 as arrays housed within the support.

In some embodiments, the microneedles are attached to an actuation mechanism. The actuation mechanism is actuated for the microneedles to pierce skin. For example, the microneedles may be connected to a spring mechanism. Thus, activation of the spring mechanism inserts the plurality of microneedles in the skin of the subject. The plurality of microneedles housed within the device may be spring-loaded such that application of force to the outside of the device deploys the microneedles. For example, the user may apply pressure to the device as it is placed on the skin to actuate the microneedles to pierce the skin.

In other embodiments, the spring mechanism may be activated upon application of the device on the skin of the subject. The microneedles may then pierce the skin of the user and begin the blood collection process. The device may include a momentary switch or release that allows the microneedles to be deployed upon actuation by the user. The momentary switch may actuate the microneedles and hold them in a piercing position without the continued application of force. In this embodiment, a second application of force on the device, once a metered quantity of blood has been collected, retracts the microneedles back into the device. In this way, in some embodiments, the activation and retraction of the microneedles of the blood collection device occur with a simple push of a button—once to deploy the microneedles, and once to retract the microneedles.

The blood collection device may include a fill indicator 909 to indicate an amount of blood being collected in the fill zone. The microneedles may stay engaged with the skin of the user and the blood collection device until the metered quantity of blood has been received by the fill zones as indicated by the fill indicator. In this embodiment, completion of collection of blood, as indicated by the fill zone, causes the microneedles to retract into the device such that the microneedles are not exposed.

In still further embodiments, the blood fill zone is housed in a removable subportion of the device and thus removable from the flexible material. The blood fill zone may be housed in a covering that includes a barcode to identify the user/patient. For example, embodiments of the invention may include a plurality of fill zones on the device, such that the fill zones together are removed from the flexible material as one piece identified by the barcode and suitable for mailing to the testing laboratory.

The device may further include a removable layer covering the adhesive layer which is removed prior to application of the device onto skin and blood extraction. The removable layer may be a plastic coating or film such as, for example, PVC, polyethylene or poly urethane, or latex.

Fill Indicator

The invention contemplates an optional fill indicator integrated into the device. A frequent challenge for remote sample collection is the collection of a sufficient quantity of sample for testing. Frequently, tests cannot be performed in the laboratory if samples fall short of the minimum quantity of specimen required for testing. Results returned as Quantity Not Sufficient (QNS) means that less than a minimum required volume or quantity of specimen was received for analyzing the panel of tests ordered. Thus, resampling is required. Further, some tests require a specimen for an initial screening, followed by further testing. Confirmatory screening and further testing require the use of another and/or different portions of the original specimen.

The fill indicator ensures that a predetermined quantity of sample, sufficient for the range of tests required, is collected and stored in the device. Thus, the fill indicator provides a visible or other indicator that a sufficient quantity of sample for testing and analysis has been collected.

The quantity of sample collected may be a pre-determined or metered quantity. The fill indicator may be incorporated into any home or remote sample collection device. For example, the fill indicator is particularly useful for remote sample collection such as with the blood collection devices of the invention. The fill indicator is capable of indicating when a metered quantity of sample has been received by the fill zones such that the user knows when the sample collection process is complete.

The device may be any of the embodiments disclosed herein. A frequent challenge for remote blood collection is the collection of a sufficient quantity of blood for testing. The invention provides for a fill indicator incorporated into devices of the invention as a visible indicator that a sufficient quantity of blood, such as a pre-determined quantity, for testing and analysis has been collected. The fill indicator may be a visual indicator that indicates when a desired quantity of blood has been received such that the fill zones on the device are adequately filled. The fill indicator ensures that the quantity of blood collection by the device is sufficient for testing while also ensuring that zones are not overfilled.

The quantity of blood required to adequately fill the fill zones may vary. For example, fill zones may require 5 to 20 μL depending on the tests to be performed. The fill indicator must ensure that enough blood is collected to fill the travel through the microchannels of the device and into the fill zones. In some embodiments, the fill indicator indicates when a predetermined quantity of blood has been collected. Thus, in non-limiting examples, the fill indicator may be calibrated to indicate when the device has collected from 50 μL to 170 μL of blood. Additionally and alternatively, the device may be calibrated such that one or more fill indicators indicate when a predetermined quantity of blood has been received in the individual fill zones.

Because the device can separate the components of blood, the fill indicator may be calibrated to indicate when a sufficient quantity of, for example, plasma has been collected by the device and/or received by the fill zone(s).

The device may be configured for a user to insert a finger or other digit into the device and hold the finger or digit in place until the fill zone receives the quantity of blood as indicated by the fill indicator. The user can then easily determine when the blood collection process is complete. The fill indicator gives a visual indication to the user that the correct quantity of blood has been received into the fill zones and prevents overfilling or underfilling of the zones. Thus, the fill indicators of the device ensure a metered quantity of blood is collected in the fill zones which prevents rejection of the sample at the diagnostic testing laboratory for insufficient quantity.

The fill indicator is capable of indicating when a metered quantity of blood has been received by the fill zones such that the user knows when the blood collection process is complete. The indicator thus ensures that a user knows how long to keep the device engaged with the skin of the finger, digit, or other area of skin, before stopping the blood collection process. In some embodiments where the user uses a standalone finger piercing apparatus, the fill indicator gives the user a visual indicator when blood dropped into the collection well has fill indicator also gives a visual indicator to the user that a sufficient quantity of blood has been collected.

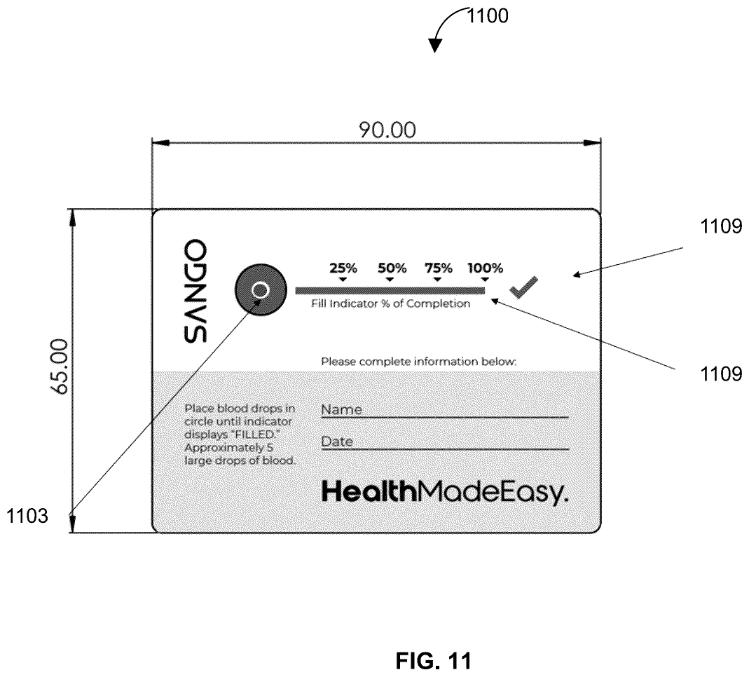

FIG. 11 illustrates an embodiment of the fill indicator 1109 integrated into the device 1100. The fill indicator 1109 may give a visual indication to the user when a metered quantity of blood has been received by the fill zones. For example, the fill indicator may be a transparent channel having a first end as a starting point and a second end as an ending point such that when blood is collected in the blood collection well 1103 and conducted from the microneedles into the fill zones, a color indicator fills the channel of the fill indicator at the first end and travels toward the second end as the blood is collected. For visual acuity, the fill indicator may be between ⅛ and ½ inch. In certain embodiments, the device may include instructions as to approximately how many drops of blood are needed to achieve the predetermined quantity of blood required for the fill zones. For example, in some embodiments, the device may include instructions directing a user to place blood drops in the circle (blood well) until indicator displays “FILLED”, which may be approximately 5 large drops of blood. The blood well may be a cone where the finger may be placed with a hanging drop of blood or in which a capillary tube may be inserted.

The fill indicator may be a plastic form that blood fills as it moves along the capillary microstructure to the fill zones. Thus, the user may watch the fill indicator as blood is drawn to ensure the blood collection is complete before stopping the blood collection. In some embodiments, once the desired quantity of blood is obtained, the plastic form may become clear and a check mark may appear to indicate the correct quantity of blood has been obtained. The fill indicator may be calibrated to indicate when collection of the predetermined quantity of blood is at 25%, 50%, 75% and 100%. As noted above, in this way, the user may watch the color indicator travel through the channel from the first end to the second end to know when the predetermined quantity of blood has been collected. The color indicator in the channel may undergo a complete color change upon collection of the predetermined quantity of blood to further visibly indicate to the user that the blood collection process is complete.

The first end of the fill indicator may be at or near the blood collection well, such that when the user places a finger in the piercing apparatus of the device, the blood collected in the blood collection well is directed to the fill indicator. The fill indicator may be calibrated to measure when a predetermined volume of blood has been directed through the capillary microstructure of the device to the fill zone(s).

The fill indicator may be a gauge with incremental markers indicating the percent completion of the blood collection process. The user may see the gauge moving as the blood is collected. For example, the indicator may be window where the user may watch an indicator, such as a colored solution or the blood itself move through the gauge to a completion marker. For example, the fill indicator may indicate when the correct quantity of blood has been received to fill one or multiple fill zones.

Additionally or alternatively, the fill indicator may be an area on the support that appears as a visible checkmark when the fill zone(s) has received a predetermined quantity of blood. The fill indicator may be a box wherein a check mark appears once the metered quantity of blood has been collected. A word indicating that a pre-determined quantity of blood has been collected into the device may appear in the box. For example, the word may be “FILLED” to indicate the fill zone or zones of the device have received a pre-determined quantity of blood.

In other embodiments, the fill indicator may be a node disposed on the support such that when a predetermined quantity of blood has been received from the microneedles into the fill zone, the node undergoes a color change. In some embodiments, the node may be substantially shaped as a button that changes color when a predetermined quantity of blood has been received by the fill zone. Thus, the fill indicator may be a button that changes color when the metered quantity of blood has been received. The button may be actuated to pop in or pop out when a predetermined quantity of blood has been received.

The fill indicator may be one or more areas on the device. For example, the device may include a fill indicator for each individual fill zone or for all fill zones collectively. In other words, each fill zone may have its own fill indicator indicating when the fill zone has received a sufficient quantity of blood. Alternatively, the device may have one fill indicator to indicate when the collective zones have each received a predetermined quantity of blood. In other embodiments, the device may comprise any number of fill indicators to ensure that when a predetermined quantity of blood has been collected, this fact is visibly indicated to the user by the fill indicator.

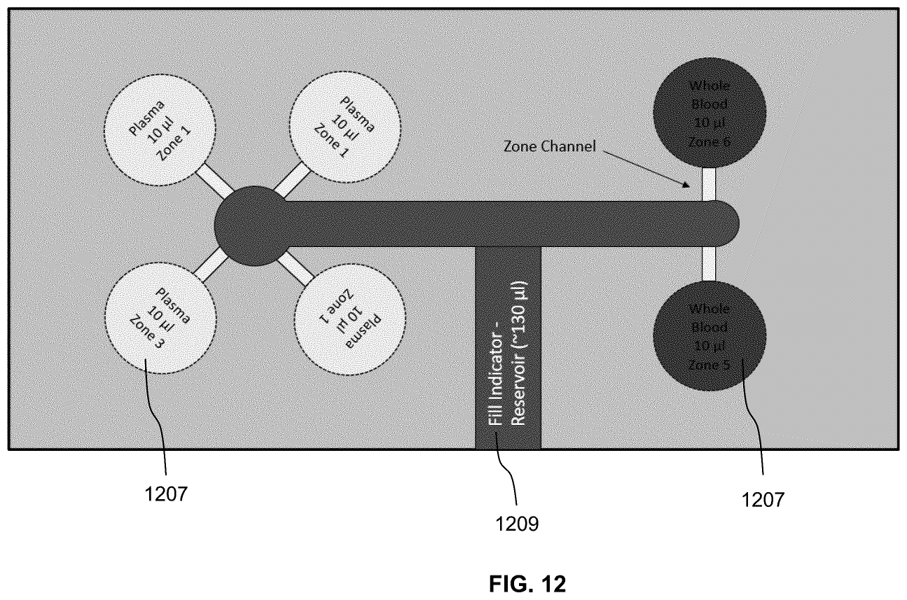

FIG. 12 further illustrates an embodiment of the device in which the fill indicator 1209 is part of a blood reservoir or blood and/or collection well 1207. The figure illustrates an embodiment of a layer of the device the houses the fill zones, channels, and reservoir. Blood from the blood reservoir may be directed through the capillary microstructure to the fill zones. The blood reservoir may be configured to collect a desired quantity of blood for directing to the fill zones. In non-limiting examples, the blood reservoir may collect from about 120 μL to about 170 μL of blood. The quantity of blood collected in the reservoir is sufficient to direct a metered volume of blood and plasma to specific fill zones within the device. Thus, the invention provides the ability to collect a sufficient quantity of blood to perform multiple tests on specimens obtained from a single device.

In certain embodiments, the fill indicator is part of a fill reservoir such that when the fill reservoir has received a predetermined quantity of blood, the fill indicator indicates that a sufficient quantity of blood has been received. Because the device can separate the components of blood, In some embodiments, the fill indicator may indicate when a sufficient quantity of, for example, plasma has been separated and received by the fill zone(s) and when a sufficient quantity of whole blood has been received by the fill zone(s).

As noted above, the fill indicator may be integrated with any of the embodiments of the devices of the invention. Aspects of the invention include a blood collection device as described herein containing a plurality of microneedles disposed with a support, at least one fill zone capable of receiving and holding a quantity of blood, a capillary microstructure extending through the support and capable of receiving and conducting the blood from the microneedles into the fill zone, and a fill indicator disposed on the support.

As discussed above, In some embodiments, the microneedles are housed in a piercing apparatus separate from the device. The user uses the microneedle piercing apparatus to pierce or puncture the skin. The finger can then be placed in the blood collection well of the device directly, or by using a capillary tube to facilitate movement of the blood from the user to the blood collection well. In this embodiment, the user places the blood in the blood collection well until the fill indicator indicates that the metered quantity of blood has been received by the fill zone or zones.

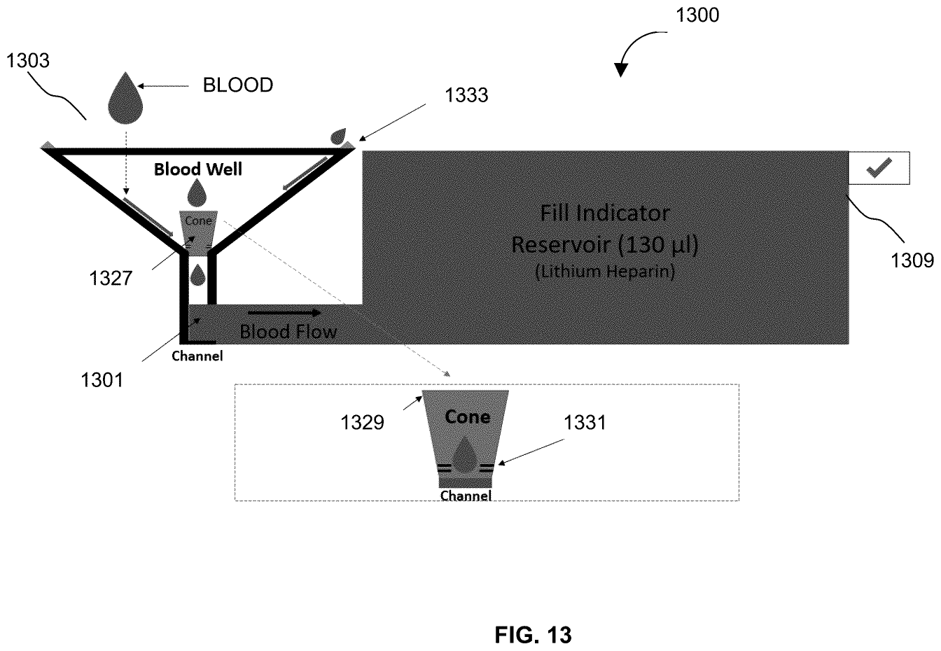

FIG. 13 illustrates a side view of one embodiment of the blood collection device 1300 of the invention showing the flow of blood from a blood collection well through the fill indicator and reservoir of the device. In this embodiment, the fill indicator is housed within the reservoir. In certain embodiments, the blood collection well 1303, the reservoir and/or the fill indicator 1309 include heparin or other anticoagulant such as lithium heparin, e.g. sodium or ammonium heparin, or ethylenediaminetetraacetic acid (EDTA) to prevent blood from clotting in the well, reservoir, or fill indicator. The fill indicator 1309 may include a box with a checkmark that appears once a predetermined quantity of blood has been collected by the reservoir. The checkmark color may be produced by a chemical color change as described above by the volume of blood reaching a space calibrated to the predetermined quantity of blood to be collected.

When the microneedles are integrated into the support of the device such as in some embodiments described above, the user keeps the finger, digit, or patch of skin engaged with the microneedles and the device until the fill indicator indicates that the predetermined quantity of blood has been collected. The fill indicator may be a visual indicator or it may be an audible indicator such as an audible pop of a node or button.

For embodiments using a standalone piercing apparatus, the user may hold the finger or other digit over the blood collection well so that blood is dropped into the blood collection well and directed to the reservoir with the integrated fill indicator. The blood may be dropped directly into the well via hanging blood drop on a fingertip or other digit or via capillary tube. In this case, the blood collection well may be substantially shaped as a funnel to allow for the flow of blood down the sides of the blood collection well to a fill indicator reservoir and into the capillary microstructure of the device. The blood collection well may have an opening or channel at the bottom of the well that directs blood flow to the capillary microstructure and/or a fill zone reservoir. This channel may itself act as a capillary tube to direct blood to the fill indicator and reservoir. The blood collection well may comprise an edge 1333 for wiping drops of blood from the finger or digit and allowing the blood to drop into the bottom of the blood collection well.

The fill indicator prevents blood from entering the fill zone once a metered quantity of blood has been received by the fill zone. In some embodiments, because blood proceeds through a channel 1301 to the fill indicator 1309, the fill indicator prevents a user from overfilling the fill zones such that no liquid blood remains in the well. The channel 1301 may, in some embodiments, act as a capillary to direct blood to the fill indicator or reservoir.

In some embodiments the well includes a cone 1327 where a finger may be placed with a drop of blood on the fingertip or via insertion of a capillary tube. The cone 1327 may have a larger opening 1329 to accommodate blood drop(s) or a capillary tube. The cone 1327 may have one or more slits 1331 on the side of the cone that allow blood to flow in from the side of the well.

Once collection is complete, the device may be dried from a period ranging from five minutes to one hour, depending on the ambient conditions under which the blood is collected. However, because the device comprises multiple layers between a top and bottom layer, with the capillary microstructure under the initial layers, the user does not have to allow for drying time before placing the device in the return pouch for safely mailing to the testing laboratory.

Aspects of the invention provide for a method of collecting blood using any of the devices of the disclosure. For example, the device may comprise a plurality of microneedles disposed on a support. The device may further comprise at least one fill zone disposed within the support to receive and hold a quantity of blood, a capillary microstructure extending through the support and capable of conducting the blood from the microneedles into the fill zone, and a fill indicator disposed on the support. As described above, the fill indicator indicates when a metered quantity of blood has been received by the fill zone from the microneedles.

The fill indicator may be a visible indicator on the support upon collection of a predetermined quantity of blood in the fill zone. For example, the fill indicator may be a transparent channel having a first end and a second end, wherein, as blood is conducted from the microneedles into the fill zone, a color indicator fills the channel at the first end and travels toward the second end such that when the fill zone has received a predetermined quantity of blood, the transparent channel undergoes a complete color change to visibly indicate the fill zone has received the predetermined quantity of blood.

Alternatively and/or additionally, the fill indicator may be area on the support that appears as a visible checkmark when the fill zone has received a predetermined quantity of blood. For example, as described above, the fill indicator may be an area on the support in which a word appears to indicate when the fill zone has received a predetermined quantity of blood. The fill indicator may be a node disposed on the support such that when a predetermined quantity of blood has been received from the microneedles into the fill zone, the node undergoes a color change. For example, the node may be substantially shaped as a button. The fill indicator may prevent blood from entering the fill zone once a metered quantity of blood has been received by the fill zone.

Blood Collection Well

The device may comprise a blood collection well for receiving the blood from the user. The blood collection well may house the array of microneedles as detailed above. Where blood enters the device may be referred to as a “well.” Where blood is collected in the fill zones may be referred to as a “zone.” The user may insert a digit into a piercing apparatus integrated into the device to pierce the skin and start the flow of blood into the collection well.

Also as discussed above, devices of the invention may include a standalone piercing apparatus. The user may use a standalone piercing apparatus disclosed in the invention to pierce the skin and then drop the blood into the blood collection well of the device.

As noted above, the blood collection well may be substantially shaped as a funnel to allow for the flow of blood down the sides of the blood collection well to a fill indicator reservoir and into the capillary microstructure of the device. The blood collection well may have an opening or channel at the bottom of the well that directs blood flow to the capillary microstructure and/or a fill zone reservoir. This channel may itself act as a capillary tube to direct blood to the fill indicator and reservoir. The blood collection well may comprise an edge for wiping drops of blood from the finger or digit and allowing the blood to drop into the bottom of the blood collection well.

For devices in which the microneedles are integrated as a standalone piercing apparatus, the blood collection well may be shaped so that blood can be dropped directly into the well for example as a blood drop hanging on the finger or digit of the user, or hanging from a capillary tube.

In some embodiments, the blood collection device includes a cone seated at the bottom of the blood collection well and fluidically connected to a channel leading to reservoir and/or fill indicator and capillary microstructure. The cone may have a larger opening to accommodate blood dropped from the finger or digit or a capillary tube. The cone may have slits or openings on the side of the cone that allow blood to flow in form the side of the well, for example, if blood is dropped or wiped such that the blood flows down the sides of the well. In this way, the cone can capture all of the blood collected in the well and direct the blood to the components of the device.

In certain embodiments, the blood collection well and material wick the blood to direct it to the reservoir, fill indicator, and capillary microstructure.

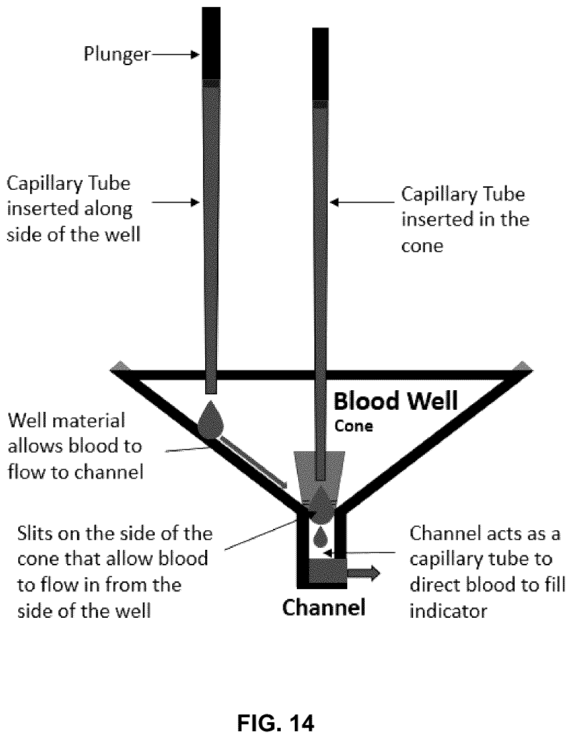

FIG. 14 illustrates one embodiment of blood flow from a capillary tube to the blood collection well. In some embodiments, the blood collection well includes a cone positioned in the bottom of the blood collection well wherein a user may use a capillary tube to direct blood from the finger, after piercing by the microneedles, to the blood collection device. The cone may have a larger opening at the top to accommodate blood drops or a capillary tube. The cone may have slits or openings on the side to allow for blood flowing down the sides of the blood collection well to enter the cone. The cone may have an opening at the bottom connected to a channel that directs the blood to the capillary microstructure of the device and a fill indicator reservoir.

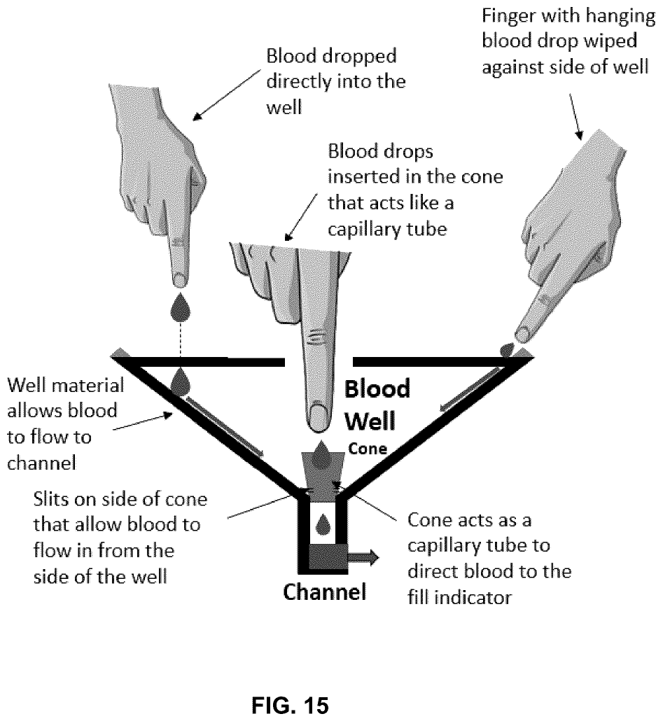

FIG. 15 illustrates one embodiment of the flow of blood from a user's finger, after piercing by the microneedles in a standalone device, to the blood well and into the channel to direct blood to the fill indicator. The reservoir may include an anticoagulant, for example lithium heparin. The blood collection well may or may not comprise a cone as described above. The design of the blood collection well allows for a user to drop blood directly into the well such that blood drops are inserted into the cone which then acts similarly to a capillary tube. In some embodiments, the material of the well allows blood to flow, for example by wicking, to the channel connecting the blood collection well with the fill indicator, reservoir, and capillary microstructure of the device. Slits on the side of the cone may be included to allow blood to flow into the cone from the side of the well.

The well may include a lip or bevel on the top portion such that a finger, with a hanging drop of blood may be wiped against the top of the blood collection well and wicked down the side of the well to the slits of the cone.

Flexible Fill Zone Configurations

The invention provides devices that can be manufactured with flexibility to have different numbers and combination of fill zones for collecting separated samples of blood and/or plasma. devices with different numbers and combinations of zones (e.g. for collecting multiple blood and/or plasma samples). This flexibility allows for multiple tests on the blood collected into the device. For example, testing of plasma collected on the device can inform testing of the whole blood component also collected on the device. For example, plasma collected in a fill zone may be tested for a virus. Then, whole blood from the same device may also be tested to inform the prescription thus eliminating the need for another sample. Additionally, collecting multiple separated samples provides for back-up samples in the event a testing error or unsatisfactory lab test.

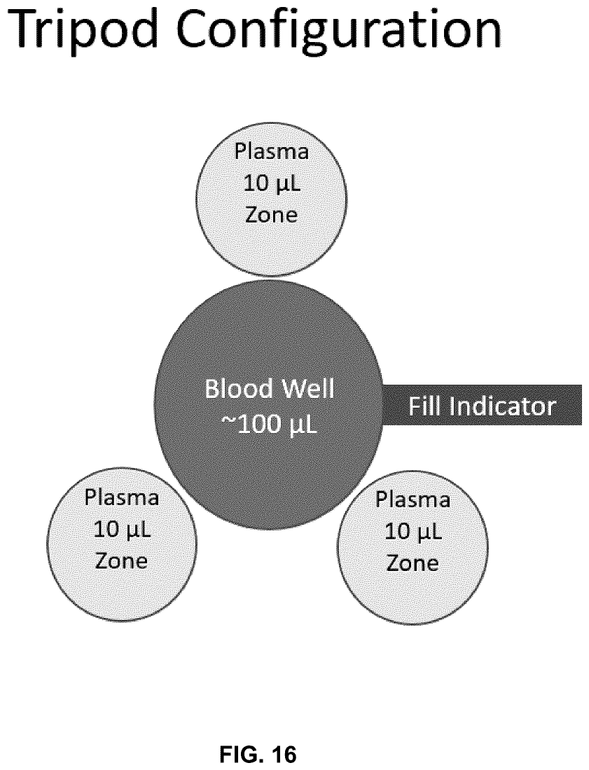

FIG. 16 illustrates an embodiment of fill zone configuration in the blood collection device of the invention wherein the blood collection well and plasma fill zones are in a tripod configuration.

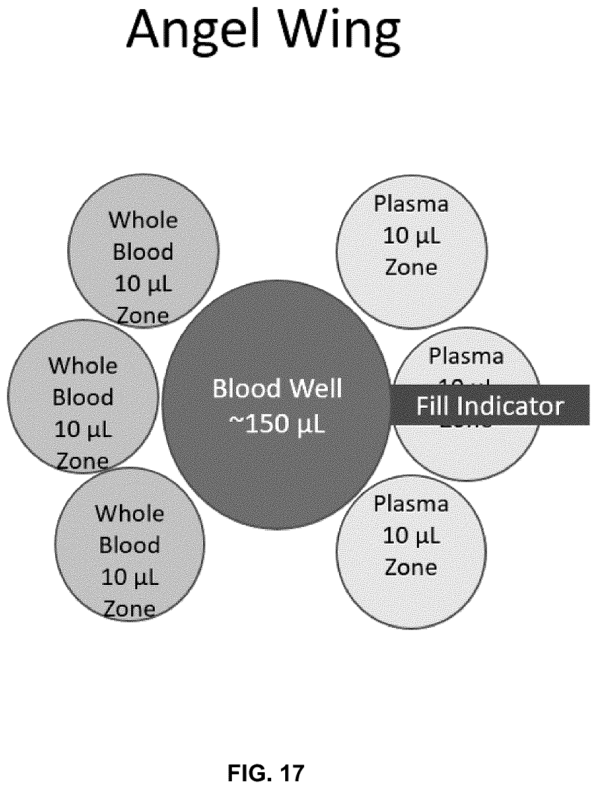

FIG. 17 illustrates another embodiment of fill zone configuration in the blood collection device of the invention wherein the blood well and fill zones, both whole blood and plasma zones, are in an angel wing configuration. The angel wing configuration may include a blood collection well at the center of the device and configured to collect about 150 μL of blood. The fill indicator may or may not be part of the blood collection well. The fill zones are configured around the blood collection well with plasma collected into fill zones on one side of the blood collection well, and whole blood collected into fill zones on the opposite side of the blood collection well.

Aspects of the invention include devices of the invention such that have a flexible ratio of whole blood collection zones and plasma collection zones. For example, the device may comprise a plurality of microneedles disposed on a support; a blood collection well disposed within the support and capable of collecting blood from the microneedles; and a capillary microstructure extending through the support and capable of receiving blood from the blood collection well, separating plasma from blood, and conducting the blood and the plasma into separate fill zones. The fill zones are capable of receiving and holding a quantity of blood and/or plasma.

The blood collection device collects a sufficient quantity of blood to separate plasma from whole blood into separate fill zones with sufficient sample quantity. For example, the blood collection well may collect about 150 μL of blood. Thus, the device may direct into the fill zones, approximately 10 μL of blood or plasma. In certain embodiments, the blood is directed into the fill zones through the capillary microstructure of the device.

In some embodiments, the device includes a substrate. For example, the device may include a blood collection well that includes a substrate that is a hydrophilic, porous media for receiving blood. For example, the porous media may be a material such as a porous composite, cellulose, acetate or any other fiber with the required porosity. For example, the substrate may coat aspects of the devices such that the substrate is functionalized to attract blood into the blood collection well, reservoir, or capillary microstructure of the device. The substrate may be, for example, a crosslinked hydrogel bound to the blood collection well.

In some embodiments, the capillary microstructure directs blood to separation media and conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

As detailed above, the support may include one or more barriers disposed on the one or more layers. The barriers may be hydrophobic wax barriers. The hydrophobic wax barriers may define one or more of a sample addition zone, the capillary microstructure, the fill zone, and the fill indicator. The layers may define a top and a bottom surface of the support.

In certain embodiments, the support may be a patterned dried blood spot support that includes one or more hydrophobic wax barriers disposed on the one or more layers. For example, the wax barriers may be formed by wax printing using a double-sided wax-transfer method to pattern the support. For example, the top and bottom designs may be printed onto laminate sheets, using a wax printer, for example a Xerox ColorQube 8580 wax printer. The various layers, for example a chromatography paper layer, may be aligned with the top and bottom designs. Alignment can be achieved by a clamp or alignment jig. The wax from the laminate sheets may be transferred to the chromatography paper using a heat press, for example a Promo Heat CS-15, to form the hydrophobic wax barriers. The hydrophobic wax barriers are patterned to define the different parts of the device.

In certain embodiments, devices of the invention are configured to have a ratio of whole blood fill zones to plasma fill zones depending on the blood tests required. For example, the device may include a capillary microstructure housed within layers of the support and arranged to separate plasma from blood and conduct blood and plasma to fill zones on the device in a predetermined ratio. For example, the ratio of blood fill zones to plasma fill zones may be from about 0:6 to about 6:0. In non-limiting examples, the ratio of blood fill zones is 0:6, 1:5, 2:4, 3:3, 4:2, 5:1, or 6:0.

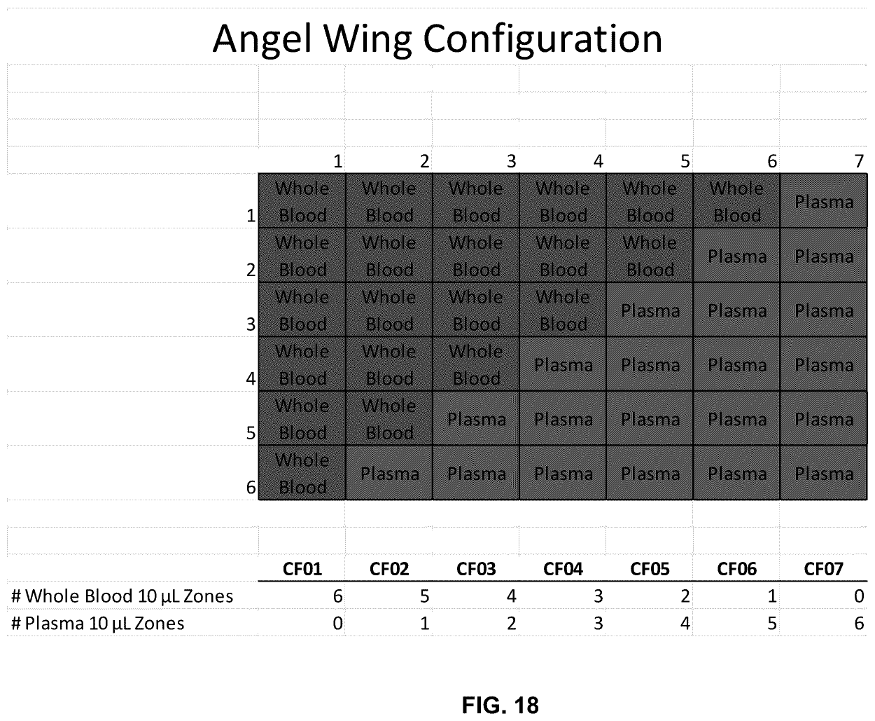

FIG. 18 illustrates the flexible embodiments of fill zone configurations of the blood collection device wherein a desired amount of whole blood and/or plasma can be configured by the arrangement of the fill zones.

As noted above, the device may have hollow or solid microneedles, or a combination thereof. The microneedles may contain a coating comprising an analgesic and/or an anticoagulant.

Components of the device are described in detail above. In some embodiments, the support is configured to allow a user to insert a finger or other digit into the device and hold the digit in place until the fill zone receives a desired quantity of blood. In some embodiments of the device, the finger or other digit is held in place by a surface that conforms to the finger and guides a placement of the finger for piercing by the microneedles.

The device may also include a fill indicator that visibly shows when the fill zone has received a pre-determined quantity of blood. In some embodiments, the support is a substantially flat surface. For example, the support may be sized and configured to easily mail back to a testing laboratory. In other embodiments, the support is flexible to allow conformation to a surface to which it is applied. In some embodiments, the support is a substantially flat surface.

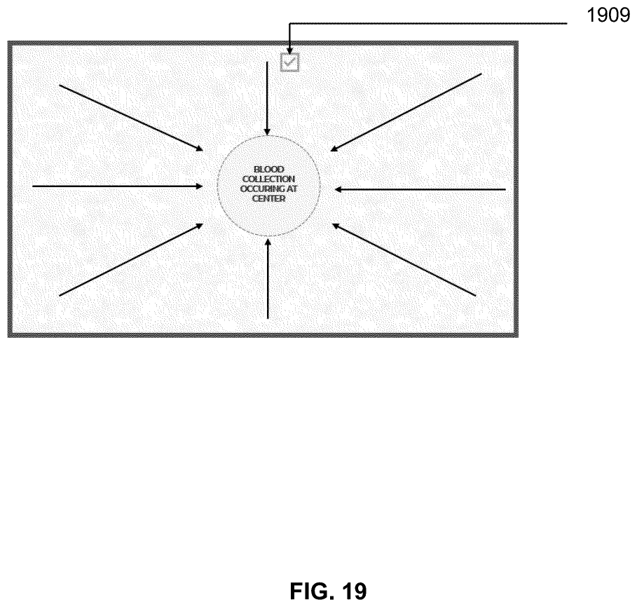

FIG. 19 illustrates an embodiment of devices of the invention in which the device includes a flexible support. The device that may be applied to skin as a patch. In this embodiment, the device is integrated into an adhesive strip. The backside, or side with the adhesive, includes the microneedles positioned so as to contact the skin of the user. As shown in FIG. 19, the blood is collected in a collection well after the device has been applied to the body of the user for collection of blood for a certain duration. As demonstrated in FIG. 19, the backside of the device, i.e., the device not in contact with the skin, has a fill indicator 1909, which may become visible and/or change color upon collection of the required quantity of the blood. In some embodiments, the blood is collected in a perforated center region by the channels.

The flexible support may be a stretchable and/or deformable material to conform to a shape of an area to which the device is applied. The support may be, for example, plastic, cellulose, cardstock, cover stock, pasteboard, paperboard, fiber board, or a cardboard such as a folding boxboard, chipboard, Kraft board, laminated board, or solid bleached or unbleached board. The support may also include a flexible mesh fabric or woven material for flexibility in the application of the device. The support may be one or more of materials that provide the balance of rigidity, flexibility, weight, and durability to house the components of the device. For example, the support may be a woven fabric, plastic, such as PVC, polyethylene or poly urethane, or latex. The flexible material may comprise a stretchable and/or deformable material to conform to a shape of an area to which the device is applied. For example, the device may comprise adhesives and channels integrated into an adhesive strip. In some embodiments, the adhesive strip is applied to a user's body, in non-limiting examples, such as an arm (including shoulder), leg, (including thighs), buttocks, or back. The adhesive strip may be applied to the skin of the body for a time period and/or until the fill indicator indicates that the metered quantity of blood has been collected. In some embodiments. The top of the strip may turn a different color to indicate when an appropriate volume of blood has been collected. The strip may be then removed and air-dried for a period of time, for example an hour, before mailing to a laboratory for analysis.

The flexible support of the blood collection device housing the plurality of microneedles is designed to conform to the shape of the body where the blood collection is applied as a patch. The flexible support conforms to the shape of the body such that the plurality of microneedles housed in the flexible patch may contact the skin. The flexible support may comprise a polymer with high elasticity and deformability. The support may vary in thickness to meet the needs of the device, such as about 5000 micrometers or less, in some embodiments from about 1000 to about 2000 micrometers, from about 1 to about 500 micrometers, and in some embodiments, from about 10 to about 200 micrometers. The flexible support may include at least one of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, Gore-Tex, plastic, plastic components, polymer, biopolymer, woven material, non-woven material, and natural material. The flexible support may also comprise a polyurethane-based film. In some embodiments, the flexible support has a shape comprising at least one of a circle, oval, ellipse, square, rectangle, triangle, diamond, butterfly, and hourglass. The invention further provides that the flexible support is larger than the area of microneedle assembly. Thus, there is an area surrounding the microneedle assembly that contains backing with no microneedles. This design makes it easier to apply the device. It also reduces the risk of unintentional contact of the microneedle assembly with the skin either during application or removal. The unintentional contact of the microneedle assembly with the skin can result in bleeding or infection. This is especially important if another person applies or removes the device as this will reduce the risk of transmitting infections between them. The microneedle assembly may be placed in the center of the backing or off center.

The device of the invention may further comprise an adhesive layer that can help facilitate the attachment of the patch to a user's skin during use. The adhesive layer typically employs an adhesive coated onto a backing material. The backing may be made of a material that is substantially impermeable to blood, such as polymers, metal foils, etc. Suitable polymers may include, for instance, polyethylene terephthalate, polyvinylchloride, polyethylene, polypropylene, polycarbonate, polyester, and so forth. The adhesive may be a pressure-sensitive adhesive. Suitable adhesives may include, for instance, solvent-based acrylic adhesives, solvent-based rubber adhesives, silicone adhesives, etc.

The flexible support may comprise one or more layers for example, a laminate layer on a top portion of the device for sealing, layers of polyester mesh, clear PET mylar membranes, polyester membranes, clear PETG membranes, asymmetrical polysulfone membranes, double-sided adhesive to affix the membranes, and a cellulose or chromatography layer or cut out. The first side, or underside, of the blood collection device may include an adhesive layer to secure the device to the skin. The adhesive layer may be covered with a removable adhesive backing such that the removable adhesive backing is removed prior to applying the device to skin. The adhesive may be, for example, an acrylate or vinyl resin, safe for adhesion of the device to skin.

One or more layers may be filter paper or chromatography paper. For example, the paper may be 100% pure cotton linter filter paper such as A-226 paper from PerkinElmer or a general qualitative filter with creped surface such as 226 from Whatman. Alternatively the filter papers may be Ahlstrom Grade 226, Munktell TFN, and Whatman 903 filter papers.

The first side, or underside, of the blood collection device may include an adhesive layer to secure the device to the skin. The adhesive layer may be covered with a removable adhesive backing such that the removable adhesive backing is removed prior to applying the device to skin. The adhesive may be, for example, an acrylate or vinyl resin, safe for adhesion of the device to skin.

As detailed above, the support may be a patterned dried blood spot support that includes one or more barriers disposed on the one or more layers. The barriers may be hydrophobic wax barriers. For example, the wax barriers may be formed by wax printing using a double-sided wax-transfer method to pattern the support. For example, the top and bottom designs may be printed onto laminate sheets, using a wax printer, for example a Xerox ColorQube 8580 wax printer. The layers, for example the chromatography layer, may be aligned with the top and bottom designs. Alignment can be achieved by a clamp or alignment jig. The wax from the laminate sheets may be transferred to the chromatography paper using a heat press, for example a Promo Heat CS-15, to form the hydrophobic wax barriers. The hydrophobic wax barriers are patterned to define the different parts of the device.

The capillary microstructure may comprise channels 801. The capillary microstructure may include a separation mechanism in the channels that retains a cellular fraction of the blood at a predetermined location on the solid support and conducts plasma to the fill zone. The separation may be achieved by virtue of the capillary structure of the device. Thus, the microstructure is capable of separating plasma and whole blood fractions into separate fill zones on the device.

In some embodiments, the capillary microstructure directs blood to separation media or separation mechanism, which conducts a cellular fraction of the blood to a predetermined location on the support and conducts plasma to the fill zone. Separation may be passive, relying on the different behavior of cells and plasma in the fluidic system. The separation media may be a separation membrane, for example, such as an asymmetric polysulfone membrane. The separation mechanism may be the capillary microstructure itself. The separation mechanism may be a gel, filter paper or particle. For example, the separation mechanism may be an interlocked micropillar scaffold provided on synthetic paper. The synthetic paper may be made from synthetic polymers to provide a polymer-based substrate. The synthetic paper may be a porous substrate with a low internal surface area designed for the use in capillary-driven lateral flow devices.

In some embodiments of the device, the blood collection channel comprises a capillary structure that carries blood away from the at least one microneedle. In a non-limiting example, blood may be carried away from the at least one microneedle through capillary action. Blood may be wicked by the microneedles to the blood collection channel and into the capillary microstructure to the fill zones. The distal portion of the blood collection channel may include a blood fill zone of a predetermined volume. For example, the fill zone may be at least 10 μL.

The microneedles housed within the device may be hollow or solid, or a combination of both. In certain embodiments, the microneedles are hollow. The device may include a plurality of microneedle arrays spaced throughout the device. The microneedles may be coated with an analgesic such as lidocaine, and/or an anticoagulant such as heparin.

In some embodiments, the device may be part of a kit. For example, a doctor, laboratory, or research study coordinator may ship the device as a kit to the user at home or to a remote location, or arrange for the device to be picked up at a designated clinic or other site. The integrated nature of the device may mean there are no ancillary supplies necessary, and instructions may be printed directly on the blood collection device.

The device may include an envelope for mailing back to the testing laboratory. The device may include a desiccant for humidity control.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A blood collection device, comprising:
a plurality of microneedles disposed on a support;
at least one fill zone disposed within the support, the fill zone capable of receiving and holding a quantity of blood; and
a capillary microstructure extending through the support and capable of conducting the blood from the microneedles into the fill zone.

2. The device of claim 1, wherein the microneedles are hollow.

3. The device of claim 1, wherein the microneedles are solid.

4. The device of claim 1, wherein the support is configured to allow a user to place a finger in contact with the device for a time sufficient to allow the fill zone to receive a desired quantity of blood.

5. The device of claim 4, wherein the finger is held in place by a surface that conforms to the finger and guides a placement of the finger for piercing by the microneedles.

6. The device of claim 1, further comprising a fill indicator that visibly shows when the fill zone has received a pre-determined quantity of blood.

7. The device of claim 1, wherein the device comprises a separation mechanism that retains a cellular fraction of the blood at a predetermined location on the support and conducts plasma to the fill zone.

8. The device of claim 7, wherein the separation mechanism is selected from a group consisting of the capillary microstructure, a gel, filter paper, and a particle.

9. The device of claim 1, wherein the capillary microstructure comprises a plurality of layers.

10. The device of claim 1, wherein the support is a substantially flat surface.

11. The device of claim 1, wherein the support is flexible to allow conformation to a surface to which it is applied.

12. The device of claim 1, wherein the microneedles contain a coating comprising an analgesic and/or an anticoagulant.

13. The device of claim 1, further comprising a capillary tube for extraction of blood from the device.

14. The device of claim 1, wherein the support further comprises a substrate.

15. The device of claim 14, wherein the substrate is a hydrophilic, porous media for receiving blood.

16. The device of claim 1, wherein the support further comprises:
one or more layers; and
one or more barriers disposed on the one or more layers.

17. The device of claim 16, wherein the barriers define one or more of a sample addition zone, the capillary microstructure, the fill zone, and the fill indicator.

18. The device of claim 16, wherein the barrier is a hydrophobic material.

19. The device of claim 16, wherein the one or more layers define a top surface and a bottom surface.

20. The device of claim 1, wherein the capillary microstructure comprises lateral distribution channels.

21. The device of claim 1, wherein the device further comprises a vacuum source sufficient to draw blood from a needle puncture site to the capillary microstructure.
